SEQUENCE LISTING (78 sequences)

SEQ ID NO: 1 - DNA, 32 bp, Synthetic construct
  source 1..32: mol_type = other DNA
cgctcggacgaacaccccgacgaaatcgagcg

SEQ ID NO: 2 - DNA, 20 bp, Synthetic construct
  source 1..20: mol_type = other DNA
gacgaacaccccgacgaaat

SEQ ID NO: 3 - DNA, 32 bp, Synthetic construct
  source 1..32: mol_type = other DNA
cgctcgccgggccgaaatcggtatgtcgagcg

SEQ ID NO: 4 - DNA, 20 bp, Synthetic construct
  source 1..20: mol_type = other DNA
ccgggccgaaatcggtatgt

SEQ ID NO: 5 - DNA, 32 bp, Synthetic construct
  source 1..32: mol_type = other DNA
cgctcggaattggctcagctggctggcgagcg

SEQ ID NO: 6 - DNA, 20 bp, Synthetic construct
  source 1..20: mol_type = other DNA
gaattggctcagctggctgg

SEQ ID NO: 7 - DNA, 41 bp, Synthetic construct
  source 1..41: mol_type = other DNA
gctcgtatggcaccggaaccacactaccgcgacccaccaca

SEQ ID NO: 8 - DNA, 40 bp, Synthetic construct
  source 1..40: mol_type = other DNA
gctcgtatggcaccggaaccacactaccgcgacccaccac

SEQ ID NO: 9 - DNA, 41 bp, Synthetic construct
  source 1..41: mol_type = other DNA
gctcgtatggcaccggaacctcactacagaaacctcaccac

SEQ ID NO: 10 - DNA, 20 bp, Synthetic construct
  source 1..20: mol_type = other DNA
atttcgtcggggtgttcgtc

SEQ ID NO: 11 - DNA, 21 bp, Synthetic construct
  source 1..21: mol_type = other DNA
tcgtagccgtacaggatctcg

SEQ ID NO: 12 - DNA, 39 bp, Synthetic construct
  source 1..39: mol_type = other DNA
gtttggccccttcagtggaatattgttgataaccgacat

SEQ ID NO: 13 - DNA, 41 bp, Synthetic construct
  source 1..41: mol_type = other DNA
gggtttggccccttcagtggaatattgttgataaccgacat

SEQ ID NO: 14 - DNA, 39 bp, Synthetic construct
  source 1..39: mol_type = other DNA
gtttggccccttcagtggaatattgtcatgttcctatca

SEQ ID NO: 15 - DNA, 39 bp, Synthetic construct
  source 1..39: mol_type = other DNA
cccttcagtggctgtggctatattgtcatgttcctatca

SEQ ID NO: 16 - DNA, 40 bp, Synthetic construct
  source 1..40: mol_type = other DNA
cccttcagtggctgtggctatattgtcatgttacctatca

SEQ ID NO: 17 - DNA, 41 bp, Synthetic construct
  source 1..41: mol_type = other DNA
cccttcagtggctgtggctatattagtcatgttacctatca

SEQ ID NO: 18 - DNA, 43 bp, Synthetic construct
  source 1..43: mol_type = other DNA
cccttcagtggctgtggctatattatcgtcatgttacctatca

SEQ ID NO: 19 - DNA, 46 bp, Synthetic construct
  source 1..46: mol_type = other DNA
cagtggctgtggcagtcatctagtacactcgtcatgttacctatca

SEQ ID NO: 20 - DNA, 34 bp, Synthetic construct
  source 1..34: mol_type = other DNA
cagtggctgtggcagtcagtcatgttacctatca

SEQ ID NO: 21 - DNA, 34 bp, Synthetic construct
  source 1..34: mol_type = other DNA
gtttggccccttcagtggatcatgttacctatca

SEQ ID NO: 22 - DNA, 39 bp, Synthetic construct
  source 1..39: mol_type = other DNA
ctcgtggacataccgattgttgttcatcagtagcgagat

SEQ ID NO: 23 - DNA, 39 bp, Synthetic construct
  source 1..39: mol_type = other DNA
ctcgtggacataccgattgttgttcatcagtggcgagat

SEQ ID NO: 24 - DNA, 33 bp, Synthetic construct
  source 1..33: mol_type = other DNA
ctcgtggacataccgattgttgttcagcgagat

SEQ ID NO: 25 - DNA, 34 bp, Synthetic construct
  source 1..34: mol_type = other DNA
ctcgtggacataccgattgttgttcaggcgagat

SEQ ID NO: 26 - DNA, 39 bp, Synthetic construct
  source 1..39: mol_type = other DNA
cccttcagtggctgtggctatattgtcatgttcctctca

SEQ ID NO: 27 - DNA, 40 bp, Synthetic construct
  source 1..40: mol_type = other DNA
cccttcagtggctgtggctatattgtcatgttacctctca

SEQ ID NO: 28 - DNA, 46 bp, Synthetic construct
  source 1..46: mol_type = other DNA
cagtggctgtggcagtcatctagtacactcgtcatgttacctctca

SEQ ID NO: 29 - DNA, 41 bp, Synthetic construct
  source 1..41: mol_type = other DNA
cgctcgtggacataccgattgttgttcatcagtagcgagat

SEQ ID NO: 30 - DNA, 41 bp, Synthetic construct
  source 1..41: mol_type = other DNA
cgctcgtggacataccgattgttgttcatcagtggcgagat

SEQ ID NO: 31 - DNA, 41 bp, Synthetic construct
  source 1..41: mol_type = other DNA
cccttcagtggctgtggctatattgtcatgttcctatcatc

SEQ ID NO: 32 - DNA, 42 bp, Synthetic construct
  source 1..42: mol_type = other DNA
cccttcagtggctgtggctatattgtcatgttacctatcatc

SEQ ID NO: 33 - DNA, 48 bp, Synthetic construct
  source 1..48: mol_type = other DNA
cagtggctgtggcagtcatctagtacactcgtcatgttacctatcatc

SEQ ID NO: 34 - DNA, 38 bp, Synthetic construct
  source 1..38: mol_type = other DNA
ttcagtggctgtggcagtattgttacattataatcatc

SEQ ID NO: 35 - DNA, 38 bp, Synthetic construct
  source 1..38: mol_type = other DNA
ttcagtggctgtggcagtattgttacattataatcacc

SEQ ID NO: 36 - DNA, 41 bp, Synthetic construct
  source 1..41: mol_type = other DNA
cccttcagtggctgtggctatattgtcatgttcctatcacc

SEQ ID NO: 37 - DNA, 40 bp, Synthetic construct
  source 1..40: mol_type = other DNA
cccttcagtggctgtggctatattgtcatgttcctatcac

SEQ ID NO: 38 - DNA, 39 bp, Synthetic construct; identical to SEQ ID NO: 15
  source 1..39: mol_type = other DNA

SEQ ID NO: 39 - DNA, 42 bp, Synthetic construct
  source 1..42: mol_type = other DNA
gccccttcagtggctgtggctatattgtcatgttacctatca

SEQ ID NO: 40 - DNA, 41 bp, Synthetic construct
  source 1..41: mol_type = other DNA
cccttcagtggctgtggctatattgtcatgttaacctatca

SEQ ID NO: 41 - DNA, 39 bp, Synthetic construct
  source 1..39: mol_type = other DNA
ccttcagtggctgtggcatatattgtcatgtttatcgta

SEQ ID NO: 42 - DNA, 39 bp, Synthetic construct
  source 1..39: mol_type = other DNA
cttcagtggctgtggcagtatattgtcatgtttatcgta

SEQ ID NO: 43 - DNA, 40 bp, Synthetic construct
  source 1..40: mol_type = other DNA
cttcagtggctgtggcagtatattgtcatgttctatcgta

SEQ ID NO: 44 - DNA, 41 bp, Synthetic construct
  source 1..41: mol_type = other DNA
cccttcagtggctgtggcagtatattgtcatgtttatcgta

SEQ ID NO: 45 - DNA, 42 bp, Synthetic construct
  source 1..42: mol_type = other DNA
cccttcagtggctgtggcagtatattgtcatgttctatcgta

SEQ ID NO: 46 - DNA, 20 bp, Synthetic construct
  source 1..20: mol_type = other DNA
ggaaatcgcagccacgttac

SEQ ID NO: 47 - DNA, 20 bp, Synthetic construct
  source 1..20: mol_type = other DNA
ttacgctcgtggacataccg

SEQ ID NO: 48 - DNA, 20 bp, Synthetic construct
  source 1..20: mol_type = other DNA
cgtaaccccagtgcgaaagt

SEQ ID NO: 49 - DNA, 18 bp, Synthetic construct
  source 1..18: mol_type = other DNA
ctcgtggacataccgatt

SEQ ID NO: 50 - DNA, 20 bp, Synthetic construct
  source 1..20: mol_type = other DNA
cacgttacgctcgtggacat

SEQ ID NO: 51 - DNA, 41 bp, Synthetic construct
  source 1..41: mol_type = other DNA
tcggcgcttgtgggtcaacctgcttgtatacacattctgga

SEQ ID NO: 52 - DNA, 39 bp, Synthetic construct
  source 1..39: mol_type = other DNA
ggcgcttgtgggtcaacctgcttgtatacacattctgga

SEQ ID NO: 53 - DNA, 42 bp, Synthetic construct
  source 1..42: mol_type = other DNA
tcggcgcttgtgggtcaacctgcttgtatacacagttctgga

SEQ ID NO: 54 - DNA, 40 bp, Synthetic construct
  source 1..40: mol_type = other DNA
ggcgcttgtgggtcaacctgcttgtatacacagttctgga

SEQ ID NO: 55 - DNA, 41 bp, Synthetic construct
  source 1..41: mol_type = other DNA
gacagaccgccgggccccagtattacagtaaattgcttgtc

SEQ ID NO: 56 - DNA, 37 bp, Synthetic construct
  source 1..37: mol_type = other DNA
acgtgacagaccgccgtattacagtaaattgcttgtc

SEQ ID NO: 57 - DNA, 38 bp, Synthetic construct
  source 1..38: mol_type = other DNA
acgtgacagaccgccgtattacagtaaattcgcttgtc

SEQ ID NO: 58 - DNA, 39 bp, Synthetic construct
  source 1..39: mol_type = other DNA
cagaccgccgggccccagtattacagtaaattgcttgtc

SEQ ID NO: 59 - DNA, 39 bp, Synthetic construct
  source 1..39: mol_type = other DNA
tcacgtgacagaccgccgtattacagtaaattgcttgtc

SEQ ID NO: 60 - DNA, 42 bp, Synthetic construct
  source 1..42: mol_type = other DNA
gacagaccgccgggccccagtattacagtaaattcgcttgtc

SEQ ID NO: 61 - DNA, 40 bp, Synthetic construct
  source 1..40: mol_type = other DNA
cagaccgccgggccccagtattacagtaaattcgcttgtc

SEQ ID NO: 62 - DNA, 40 bp, Synthetic construct
  source 1..40: mol_type = other DNA
tcacgtgacagaccgccgtattacagtaaattcgcttgtc

SEQ ID NO: 63 - DNA, 41 bp, Synthetic construct
  source 1..41: mol_type = other DNA
gacagaccgccgggccccagtattacagtaaattgcttgta

SEQ ID NO: 64 - DNA, 37 bp, Synthetic construct
  source 1..37: mol_type = other DNA
acgtgacagaccgccgtattacagtaaattgcttgta

SEQ ID NO: 65 - DNA, 38 bp, Synthetic construct
  source 1..38: mol_type = other DNA
acgtgacagaccgccgtattacagtaaattcgcttgta

SEQ ID NO: 66 - DNA, 39 bp, Synthetic construct
  source 1..39: mol_type = other DNA
cagaccgccgggccccagtattacagtaaattgcttgta

SEQ ID NO: 67 - DNA, 39 bp, Synthetic construct
  source 1..39: mol_type = other DNA
tcacgtgacagaccgccgtattacagtaaattgcttgta

SEQ ID NO: 68 - DNA, 42 bp, Synthetic construct
  source 1..42: mol_type = other DNA
gacagaccgccgggccccagtattacagtaaattcgcttgta

SEQ ID NO: 69 - DNA, 40 bp, Synthetic construct
  source 1..40: mol_type = other DNA
cagaccgccgggccccagtattacagtaaattcgcttgta

SEQ ID NO: 70 - DNA, 40 bp, Synthetic construct
  source 1..40: mol_type = other DNA
tcacgtgacagaccgccgtattacagtaaattcgcttgta

SEQ ID NO: 71 - DNA, 41 bp, Synthetic construct
  source 1..41: mol_type = other DNA
cccggcacgctcacgtgacatgttatactaaattagcgcca

SEQ ID NO: 72 - DNA, 41 bp, Synthetic construct
  source 1..41: mol_type = other DNA
cggcacgctcacgtgacagatgttatactaaattagcgcca

SEQ ID NO: 73 - DNA, 39 bp, Synthetic construct
  source 1..39: mol_type = other DNA
cggcacgctcacgtgacatgttatactaaattagcgcca

SEQ ID NO: 74 - DNA, 42 bp, Synthetic construct
  source 1..42: mol_type = other DNA
cccggcacgctcacgtgacatgttatactaaattcagcgcca

SEQ ID NO: 75 - DNA, 42 bp, Synthetic construct
  source 1..42: mol_type = other DNA
cggcacgctcacgtgacagatgttatactaaattcagcgcca

SEQ ID NO: 76 - DNA, 40 bp, Synthetic construct
  source 1..40: mol_type = other DNA
cggcacgctcacgtgacatgttatactaaattcagcgcca

SEQ ID NO: 77 - DNA, 20 bp, Synthetic construct
  source 1..20: mol_type = other DNA
gaggcgatcacaccgcagac

SEQ ID NO: 78 - DNA, 21 bp, Synthetic construct
  source 1..21: mol_type = other DNA
gccgcgatcaaggagttcttc

(19) United States
(12) Patent Application Publication
(10) Pub. No.: US 2025/0034659 A1
(43) Pub. Date: Jan. 30, 2025

(54) DETECTION OF MULTIDRUG-RESISTANT MYCOBACTERIUM TUBERCULOSIS USING SUPERSELECTIVE PRIMER-BASED REAL-TIME PCR ASSAYS

(71) Applicant: Rutgers, The State University of New Jersey, New Brunswick, NJ (US)

(72) Inventors: Salvatore A. E. Marras, Roselle Park, NJ (US); Selvakumar Subbian, Newark, NJ (US); Christopher Vinnard, Newark, NJ (US); Anshika Narang, Sunnyvale, CA (US)

(73) Assignee: Rutgers, The State University of New Jersey, New Brunswick, NJ (US)

(21) Appl. No.: 18/785,635

(22) Filed: Jul. 26, 2024

Related U.S. Application Data
(60) Provisional application No. 63/515,902, filed on Jul. 27, 2023.

(51) Int. Cl. C12Q 1/689; C12Q 1/6825; C12Q 1/686

(52) U.S. Cl. CPC C12Q 1/689; C12Q 1/6825; C12Q 1/686; C12Q 2600/156

(57) ABSTRACT

This disclosure relates to assays and reagents for the detection of drug-resistance mutations in Mycobacterium. tuberculosis or of multidrug-resistant Mycobacterium tuberculosis.

### CROSS-REFERENCE TO RELATED APPLICATIONS

This application claims the benefit under 35 U.S.C. § 119 (e) of the earlier filing date of U.S. Provisional Patent No. 63/515,902, filed Jul. 27, 2023, which is hereby incorporated by reference in its entirety.

### GOVERNMENT INTERESTS

This invention was made with government support under R21AI146820 awarded by the National Institutes of Health. The government has certain rights in the invention.

### REFERENCE TO AN ELECTRONIC SEQUENCE LISTING

The contents of the electronic sequence listing (096747.00491_SeqList.xml; Size: 70,998 bytes; and Date of Creation: Jul. 16, 2024) are herein incorporated by reference in its entirety.

### FIELD OF THE INVENTION

This disclosure relates to assays and reagents for the detection of drug-resistance mutations in Mycobacterium tuberculosis or of multidrug-resistant Mycobacterium tuberculosis.

### BACKGROUND

In 2020, tuberculosis (TB) was the second leading infectious killer disease worldwide after COVID-19, with a disease burden shared across all countries, genders, and age groups. In 2020, an estimated 10 million people fell ill with TB, and 1.5 million people died [1]. Multidrug-resistant TB (MDR-TB), defined as resistance to isoniazid (INH) and rifampin (RIF), remains a serious public health threat worldwide. Preventing the emergence of bacterial drug resistance is a critical component of TB elimination efforts, with the World Health Organization (WHO) aiming to reduce 95% of TB-attributable deaths by 2035 [1].

The standard regimen of the directly observed therapy short course (DOTS) strategy for global TB control includes 4 months of treatment with INH, RIF, pyrazinamide (PZA), and ethambutol (EMB), followed by 2 months of INH and RIF [1]. Single-nucleotide polymorphisms (SNPs) at a handful of genetic loci (known as hot spots) are responsible for the development of the majority of drug resistance in M. tuberculosis [2]. MDR-TB is thought to develop first with the selection of INH-resistant mutant M. tuberculosis strains, followed by the selection of RIF resistance among these mutants [3]. Treatment of MDR-TB requires the use of more toxic, more expensive, and less effective second-line drugs administered for a prolonged time (more than a year) [4], although shorter MDR treatment regimens have been endorsed by WHO [5].

Acquired drug resistance in TB occurs via the selection of spontaneous mutants in a wild-type (WT) background with a spontaneous mutation rate of 10-8 to 10-9 per round of M. tuberculosis replication [6]. With a sufficiently large M. tuberculosis population in infected tissue (greater than 109 organisms) [7], spontaneous mutants with resistance to a single drug are likely to be present, which could then be selected by inadequate drug pressure [3]. Heteroresistant TB is defined as a disease in which drug-resistant and drug-susceptible M. tuberculosis strains co-exist in the same patient [8-10]. In the conventional proportional drug sensitivity tests (pDST), heteroresistance is defined as 1-99% bacterial colony growth on drug-containing media [11].

Current TB diagnostic methods are insensitive in detecting low levels of mutant M. tuberculosis in a WT background, with lower limits of quantification ranging between 1 and 10% for standard methods, such as line probe assays [12,13] and mycobacteria growth indicator tubes (MGIT) [14]. Critically, the major limitation of traditional polymerase chain reaction (PCR) methodology is the inability to detect rare mutant sequences in a background of abundant WT DNA. With recent advances, such as digital PCR and next-generation sequencing [15], the limit of detection of rare mutants may approach the 0.1% threshold (corresponding to the ratio of mutant DNA/WT DNA), which remains far above the level required to detect the baseline presence of heteroresistant strains in clinical samples or dynamic changes in the emergence of drug-resistant M. tuberculosis [7]. Even with the limitations of current tools, the clinical impact of heteroresistant TB is increasingly recognized [8,11,14,16]. A recent study reported the weighted pooled prevalence of INH heteroresistance (from 19 studies) to be 5% and RIF heteroresistance (from 17 studies) to be 7% from 2001 to 2020 [17]. Among South African TB patients, heteroresistant infections identified by the mycobacterial interspersed repetitive units variable number of tandem repeat (MIRU-VNTR) genotyping were associated with a 90% increase in the odds of delayed sputum conversion after 2 months of treatment [18], a critical treatment endpoint for TB control programs (and related to the risk of ongoing transmission in the community). Heteroresistant M. tuberculosis infection can lead to a false negative rapid nucleic acid test for RIF resistance [19,20] and contributes to variable treatment responses observed in different anatomic regions of infected lungs [21]. Most worrisome, patients with clinically undetected heteroresistance can progress to MDR-TB in the face of first-line treatment pressure [22], either due to formulation differences or intra-individual pharmacokinetic variability [24]. All of these mechanisms contribute to the failure of TB control efforts through inadequate treatment and the further spread of drug-resistant M. tuberculosis strains [3].

Thus, there is a need for assays and reagents for the detection of multidrug-resistant M. tuberculosis, and in particular heteroresistance.

### SUMMARY

This disclosure addresses the need mentioned above in a number of aspects.

In one aspect, the disclosure provides a SuperSelective primer specific for a mutant target sequence of one selected from the group consisting of a katG gene, an inhA promoter, or an rpoB gene of M. tuberculosis. In one embodiment, the SuperSelective primer comprises one or more target-complementary sequences that are at least 85% (e.g., 90%, 91%, 92%, 93%, 94%, 95%, 96%, 97%, 98%, 99%, or 100%) identical to one selected from those listed in Table 2 below or the group consisting of SEQ ID NOs: 7-78. Examples of the target-complementary sequences include an anchor sequence and/or foot sequence disclosed herein.

In one embodiment, the SuperSelective primer comprises, in the 5′ to 3′ direction, the following three contiguous nucleic acid sequences: (i) an anchor sequence capable of forming a hybrid with the gene that may contain the mutant target sequence; (ii) a bridge sequence that is not complementary to either the mutant target sequence or the wild-type target sequence thereof; and (iii) a foot sequence that is perfectly complementary to the mutant target sequence and mismatched to the wild-type target sequence. If the anchor sequence and the foot sequence are hybridized either to the mutant target sequence or to the wild-type target sequence, there is in the target sequence an intervening sequence that does not hybridize to the primer's bridge sequence, and the bridge sequence and the intervening sequence together create a single-stranded bubble in the resulting hybrid.

The anchor sequence or the hybrid formed with the mutant target sequence can be 10-50, 15-30, 15-40, or 20-30 nucleotides in length. The bridge sequence can be at least 5 nucleotides in length. The foot sequence can be 5-20, 5-15, 6-12, 7-14, 7-9, 8-10, 5-7, or 6-7 nucleotides in length. The intervening sequence can be at least 5 nucleotides in length. The bridge sequence and the intervening sequence can be of equal length or of different lengths. The bubble can have a circumference of 10-70, 15-50, 18-50, 16-52, or 28-44, nucleotides.

In one embodiment, the foot sequence is mismatched to the wild-type target sequence by at least one of the 3′ nucleotide or the 3′ penultimate nucleotide of the foot sequence.

In one embodiment, the mutant target sequence comprises a mutation selected from the group consisting of katG AGC→ACA, katG AGC→ACC, inhA-8T→A, inhA-15C→T, inhA-17G→T, rpoB GAC→GTC, rpoB CAC→GAC, rpoB CAC→TAC and rpoB TCG→TTG.

In a second aspect, the disclosure provides a method for detecting drug resistance in M. tuberculosis (and in particular for detecting heteroresistance) or for detecting drug resistant 15 M. tuberculosis. The method comprises (A) providing a first primer pair specific for a first target segment of a first region selected from the group consisting of a katG gene, an inhA promoter, and an rpoB gene, said first primer pair comprising a first forward primer and a first reverse primer; (B) forming a reaction mixture comprising (i) the first primer pair and (ii) a sample comprising a nucleic acid of M. tuberculosis; (C) amplifying the first target segment in the reaction mixture with the first primer pair to generate a first amplicon, and (D) detecting the first amplicon, whereby the presence of the first amplicon is indicative of the drug resistance or drug-resistant M. tuberculosis in the sample. The first forward primer or the first reverse primer may comprise the SuperSelective primer described above.

In one embodiment, amplification is carried out by a polymerase chain reaction (PCR). The PCR can be a real-time PCR or a quantitative PCR or a non-symmetric PCR.

In one embodiment, amplification is carried out in the presence of a detection agent. Examples of the detection agent include a double-stranded DNA binding dye or a probe. In one embodiment, the probe can be labelled with a fluorophore. In one embodiment, the probe is labelled with a fluorophore and a quencher. Any suitable fluorophore or/and quencher can be used. Examples of fluorophores can be selected from the group consisting of FAM, Cal Fluor Red, Quasar 670, fluorescein, cyanine 3, cyanine 5, Texas Red, and TAMRA. Examples of the quencher can be selected from the group consisting of BHQ1, BHQ2, and DABCYL.

In some embodiments, the method further comprises amplifying a second target segment with a second primer pair to generate a second amplicon. The second primer pair comprises a second forward primer and a second reverse primer. This further amplification and the amplification described above can be done in the same reaction mixture (i.e., a multiplexed assay or method) or in two different reaction mixtures. The second primer pair can be specific for a segment of a second region selected from the group consisting of the katG gene, the inhA promoter, and the rpoB gene. In one embodiment, the second target segment is different from the first target segment. The second forward primer or the second reverse primer may comprise the SuperSelective primer described above.

In some embodiments, the resistance is to a drug selected from the group consisting of isoniazid, rifampicin, the fluoroquinolone class of drugs, amikacin, kanamycin, capreomycin, and ethambutol.

In a third aspect, the disclosure features a probe comprising the sequence of one selected from the group consisting of SEQ ID NOs: 1-6. In some embodiments, the probe is labeled as described above.

In a fourth aspect, the disclosure provides a reaction mixture composition or a kit comprising one or more of the SuperSelective primers described herein. The reaction mixture composition or kit may further comprise one or more of: (i) an amplification primer for the katG gene, the inhA promoter, or the rpoB gene disclosed herein; (ii) a probe specific for the katG gene, the inhA promoter, or the rpoB gene; (iii) a DNA polymerase; (iv) nucleotide triphosphates; (v) a double-stranded DNA binding dye; (vi) a selectivity-enhancing reagent, and (vii) a buffer. In some embodiments, the selectivity enhancing reagent can be a Hofmeister salt, such as tetramethylammonium chloride (TMAC) or bis-tetramethylammonium oxalate.

The details of one or more embodiments of the disclosure are set forth in the description below. Other features, objectives, and advantages of the disclosure will be apparent from the description and from the claims.

### BRIEF DESCRIPTION OF THE DRAWINGS

FIGS. 1A and 1B show SuperSelective primer-based detection of katG S315T AGC→ACC mutant sequences in the presence of abundant wild-type sequences. (FIG. 1A) shows the results of RT-PCR assays to detect katG S315T AGC→ACC mutations with a fluorescein (FAM)-labeled molecular beacon probe utilizing the SuperSelective primer 20-14/14-5:1:0 with genomic DNA (gDNA) of Mycobacterium tuberculosis as a template. The figure shows amplification of 10⁵ (black solid), 10⁴ (black dotted), 10³ (black dashed), 10² (dark grey solid), 50 (dark grey dotted), 25 (dark grey dashed), 10 (grey solid), 1 (grey dotted) copies of mutant DNA template in a background of 10⁴ copies of wild-type (WT) DNA template; 10⁴ copies of WT DNA alone (grey dashed) and nuclease-free water (light grey solid) were used as controls. (FIG. 1B) shows the threshold cycle measured for each reaction that contained mutant templates is plotted as a function of the logarithm of the number of mutant templates initially present in each reaction.

FIGS. 2A, 2B, and 2C show the results of multiplex RT-PCR assays to detect katG S315T AGC→ACC, inhA-15 C→T, and rpoB S531L TCG→TTG mutations utilizing the SuperSelective primers 20-14/14-5:1:0, 18-14/13-7:1:0, and 20-14/13-7:1:0, respectively, with mutant plasmid DNA (pDNA) used as a template. Molecular beacon probes labeled with fluorescein (FAM), Cal Fluor Red 610 (CFR610), and Quasar 670 (Q670) were used to detect the amplification signals. The figure illustrates amplification of 10⁵ (black solid), 10⁴ (black dotted), 10³ (black dashed), 10² (dark grey solid), 50 (dark grey dotted), 25 (dark grey dashed), 10 (grey solid), and 1 (grey dotted) copies of mutant DNA template in a background of 10⁴ copies of wild-type DNA template; 10⁴ copies of wild-type DNA (grey dashed) and nuclease-free water (light grey solid) were used as controls. The figure shows amplification peaks of (FIG. 2A) katG S315T AGC→ACC pDNA detected in the FAM channel, (FIG. 2B) inhA-15 C→T pDNA detected in the CFR610 channel, and (FIG. 2C) rpoB S531L TCG→TTG detected in the Q670 channel. There was no amplification in the non-specific channels; hence, that data has been excluded from these graphs.

FIGS. 3A and 3B show the structure and principle of operation of SuperSelective primers. (FIG. 3A) Structure of a SuperSelective primer for detecting katG S315T (AGC→ACA) mutant sequences in the presence of katG wild-type sequences. (FIG. 3B) Principle of operation of SuperSelective primers. The selective step occurs only when a SuperSelective primer hybridizes to a DNA (−) template fragment present in the sample. Due to the small size of the foot sequence, the probability of initiation of a (+) amplicon is significantly greater if the target sequence of the foot in the (−) template fragment is a completely complementary mutant (×) sequence than if the target sequence of the foot in the (−) template fragment is a mismatched WT sequence. Suppose (+) amplicon synthesis does occur. In that case, the resulting (+) amplicon serves as a template for a conventional reverse primer and is efficiently copied during the next thermal cycle, generating a (−) amplicon in which the complement of the unique bridge sequence that was present in the SuperSelective primer is substituted for the intervening sequence that was present in the original (−) template fragment. As a result, in subsequent thermal cycles, the entire SuperSelective primer sequence is complementary to the (−) amplicon strands, and exponential amplification occurs efficiently and can be followed in real time.

FIGS. 4A and 4B show the effect of an increase in the length of the foot and intervening sequence of a SuperSelective primer on its performance. The plot shows the results of PCR assays for the detection of inhA promoter-15 mutation using 10⁵ copies of mutant plasmid DNA (pDNA) (solid lines) and 10⁵ copies of WT pDNA (dotted lines). (FIG. 4A) Comparison of threshold cycles with SuperSelective primer inh15_SSP3 (18-14/14-6:1:0) (black) and inh15_SSP4 (18-14/13-7:1:0) (grey) with foot lengths of 7 nucleotides and 8 nucleotides, respectively. (FIG. 4B) Comparison of threshold cycles with SuperSelective primer inh15_SSP3 (18-14/14-6:1:0) (red) and inh15_SSP2 (18-14/21-6:1:0) (grey) with intervening sequence lengths 14 nucleotides and 21 nucleotides, respectively.

FIGS. 5A and 5B show the efficiency of SuperSelective primer designs with a symmetric and an asymmetric bubble under different annealing temperatures. (FIG. 5A) inh15_SSP8 (18-8/8-7:1:0)-symmetric bubble (FIG. 5B) inh15_SSP7 (18-20/8-7:1:0)-asymmetric bubble. The plots show the results of PCR assays carried out for the detection of inhA promoter-15 mutation using 10⁵ copies of mutant plasmid DNA (pDNA) (grey) and 10⁵ copies of wild-type pDNA (black) at an annealing temperature of 60° C. (Solid lines), 64° C. (Broken lines), and 66° C. (Dotted lines).

FIG. 6 shows the fluorescence signal intensity of an inhA probe at different molecular beacon concentrations. The plot shows the amplification of 10⁵ copies of mutant target DNA templates using SuperSelective primer inh8_SSP1 (18-14/14-6:1:0) and a conventional reverse primer for the detection of inhA promoter mutation-8T→A at molecular beacon probe concentrations of 0.25 μM (solid), 0.125 μM (dashed), and 0.1 μM (dotted). There was no significant difference between the signal intensity with 0.1 μM, and 0.125 μM probe; however, 0.25 μM probe resulted in signals with the highest fluorescence intensity.

FIG. 7 shows the fluorescence signal intensity of an rpoB probe in asymmetric PCR. The plot shows the amplification of 10⁵ copies of rpoB D516V GAC→GTC mutant template with SuperSelective primer rpoB516_SSP4 (18-14/13-7:1:0) and a conventional reverse primer in the ratios of 1:1, 1:5, and 1:10. Asymmetric PCR with the forward primer 0.1 μM, the reverse primer 0.5 μM (solid), and 1 μM (dashed) resulted in a higher fluorescence intensity as compared to symmetric PCR with the forward and reverse primer both at 0.1 μM (dotted). However, there was no significant difference in fluorescence intensity with the reverse primer at 0.5 μM and at 1 μM.

### DETAILED DESCRIPTION OF THE INVENTION

This disclosure relates to assays and reagents for the detection of multidrug-resistant tuberculosis.

The emergence of drug-resistant tuberculosis is a significant global health issue. The presence of heteroresistant Mycobacterium tuberculosis is critical to developing fully drug-resistant tuberculosis cases. The currently available molecular techniques may detect one copy of mutant bacterial genomic DNA in the presence of about 1-1,000 copies of wild-type M. tuberculosis DNA. To improve the limit of heteroresistance detection, the inventors developed SuperSelective primer-based real-time PCR assays, which, by their unique assay design, enable selective and exponential amplification of selected point mutations in the presence of abundant wild-type DNA. SuperSelective primers were designed to detect genetic mutations associated with M. tuberculosis resistance to the anti-tuberculosis drugs isoniazid and rifampin. The efficiency of the assay in detecting heteroresistant M. tuberculosis strains was evaluated using genomic DNA isolated from laboratory strains, and clinical isolates from the sputum of tuberculosis patients. As disclosed herein, the assays detected heteroresistant mutations with a specificity of 100% in a background of up to 10⁴ copies of wild-type M. tuberculosis genomic DNA, corresponding to a detection limit of 0.01%. Therefore, SuperSelective primer-based RT-PCR assays are an ultrasensitive tool that can efficiently diagnose heteroresistant tuberculosis in clinical specimens, and they therefore can contribute to understanding drug resistance mechanisms. This approach can improve the management of antimicrobial resistance in tuberculosis and other infectious diseases.

This application discloses a revolutionary new approach in PCR primer design, called “SuperSelective PCR primers” [25], for ultrasensitive detection of drug-resistant M. tuberculosis bacilli in an abundant background of wild-type M. tuberculosis. This innovation in PCR primer design and the demonstration of these primers in RT-PCR assays provide a significant technological advancement toward detecting rare drug-resistant mutants in tuberculosis and other infectious diseases. SuperSelective primers innovate by separating a relatively long “anchor” sequence targeting the gene of interest and a short “foot” sequence selective for the point mutation (i.e., the interrogating nucleotide). In between, there is a “bridge” sequence, which is non-binding to the target DNA sequence. When the SuperSelective primer is hybridized to the template molecule, the bridge sequence in the primer and the corresponding template sequence form a single-stranded “bubble” region that functionally separates the efficient formation of the anchor hybrid from the selective formation of the foot hybrid [25]. Both the anchor (corresponding to the target gene) and the foot sequence (which contains an “interrogating nucleotide”) must bind to the DNA fragment for amplification to proceed. The use of SuperSelective PCR primers in RT-PCR overcomes the limitations of other primer-based approaches, such as amplification refractory mutation system (ARMS) primers [26], dual-priming oligonucleotide (DPO) primers [27], hairpin primers [28], and PlexPrimers [29], which are either not sufficiently sensitive to detect extremely rare mutants, not compatible with real-time PCR due to the presence of unnatural nucleotides in their sequence, or have not been shown to enable quantitative determinations in multiplex real-time PCR assays when different target mutations occur in the same codon. By relying on standard PCR equipment and reagents, SuperSelective PCR methods can be readily adopted by any laboratory equipped to perform conventional PCR, supporting implementation in settings with medium and high burdens of TB disease.

### SuperSelective PCR Primers

As used herein, a SuperSelective primer is an allele-discriminating multi-part primer structured so that it enables the detection of as few as ten copies of rare target sequences in the presence of 10,000 copies of a closely related sequence that differs by as little as a single base pair when said primer is used as the limiting primer in a PCR amplification. A SuperSelective primer has a sequence comprising, in the 5′ to 3′ direction, the following three contiguous nucleic acid sequences (e.g., DNA sequences) that are copied by extension of the other primer: an anchor sequence, a unique bridge sequence, and a unique foot sequence.

The anchor sequence is sufficiently long so that it is able to hybridize with mutant target sequences and with its wild-type sequence during primer annealing, typically a length in the range of 15-40 nucleotides, often 20-30 nucleotides.

The bridge sequence can be at least six nucleotides long and does not hybridize during primer annealing to the primer's intended target sequence or to any other closely related sequence.

The foot sequence can be 6 to 12 nucleotides long and is perfectly complementary to the intended DNA target sequence (e.g., mutant target sequences), but mismatches a closely related sequences (e.g., a wild-type sequence) by one or more nucleotides (interrogating nucleotide or nucleotides). In some embodiments, the interrogating nucleotide is the 3′-terminal nucleotide or the 3′-penultimate nucleotide.

A SuperSelective primer may also have one or more additional structural and functional characteristics in a polymerase chain reaction (PCR) amplification and detection assay as described in WO2021067527, WO2017176852, and WO2014124290, the content of which are incorporated by reference in their entireties.

For example, if the anchor sequence and the foot sequence are both hybridized to the primer's intended target sequence, the primer-target hybrid comprises in the 5′ to 3′ direction of the primer: an anchor-target hybrid, a single-stranded bubble, and a foot-target hybrid. The bubble can have a circumference of 18 to 50 nucleotides and includes an intervening sequence in the target DNA sequence that is at least eight-nucleotides long and does not hybridize to the bridge sequence during primer annealing.

The bubble isolates the foot-target hybrid from the anchor-target hybrid, and the isolated foot-target hybrid is a weak hybrid that makes copying the intended target DNA sequence unlikely as evidenced by a delay of at least two, preferably at least five, cycles in the threshold value (Ct) as compared to the Ct that would occur using a conventional primer that is free of any bridge DNA sequence.

The probability that during PCR amplification the multi-part primer will initiate copying of any closely related mutant target DNA sequence or the related wild-type target DNA sequence can be at least 1,000 times lower than the probability of initiating copying of its intended target sequence, as evidenced by a difference in threshold values (ΔCt) of at least ten thermal cycles.

The multi-part primer allows one to generate an amplicon strand that has bridge and foot sequences that are perfectly complementary to the amplicon strand's complementary strand.

The length and sequence of the bridge sequence of each multi-part primer, together with the length of the intervening sequence of its intended target sequence, can result in a threshold value (Ct) observed for a sample containing only ten copies of its intended target DNA sequence that will occur within 40-65, or preferably 55, cycles of exponential amplification and will be at least two cycles less than the Ct observed from a sample containing no copies.

As used herein, an allele-discriminating “hairpin” primer is a stem-loop oligonucleotide that, like a molecular beacon probe, contains a single-stranded region (the “loop”) flanked by complementary sequences (“arms”) that hybridize to one another to form a double-stranded region (“stem”). The loop and the 3′ arm of a hairpin primer are sufficiently complementary to the intended target sequence to hybridize thereto under primer-annealing conditions and to initiate copying. An allele-discriminating hairpin primer contains an interrogating nucleotide at or near the middle of the loop sequence.

An ARMS primer is a conventional primer that is allele-discriminating, because its 3′-terminal nucleotide is an interrogating nucleotide. An ARMS primer may include a deliberately introduced nucleotide near its 3′ end that is mismatched both to the intended target sequence and to the unintended target sequence to destabilize the primer and increase its allele discrimination.

A “conventional” primer is a single-stranded oligonucleotide that is 15-40 nucleotides in length, more usually 20-30 nucleotides in length, and that is perfectly complementary to the intended target. Any of several computer programs are commonly used to design conventional PCR primers.

Our convention for describing a primer pair is to refer to the limiting primer as the “forward” primer that is complementary to the (−) template strand of the target, and to refer to the excess primer as the “reverse” primer that is complementary to the (+) template strand of the target. It will be understood that the limiting primer may be complementary to the (+) strand, and the excess primer may be complementary to the (−) strand.

As disclosed herein, SuperSelective PCR primers were successfully used for detecting RIF and INH heteroresistance in TB with a detection limit of 0.01% for the mutant DNA/WT DNA ratio. The method shows a 10-fold improvement in detecting mutant gene copies in the presence of abundant wild-type genes of the M. tuberculosis genome over the most sensitive methods reported to date, such as digital PCR [10].

In some examples, nine SuperSelective primer designs are disclosed. These designs, when used under optimized RT-PCR assay conditions, can detect nine mutations commonly associated with RIF and INH resistance in TB disease, including katG (S315T, AGC→ACA, and AGC→ACC), inhA promoter (−8T→A, −15C→T, and −17G→T), and rpoB (D516V, GAC→GTC; H526D, CAC→GAC; H526Y, CAC→TAC; and S531L, TCG-TTG). After generating primers for mutations associated with resistance to INH and RIF, the inventors performed clinical validation of the approach utilizing M. tuberculosis DNA isolated from sputum samples obtained from TB patients, which included a mixture of drug-sensitive and drug-resistant cases. While the SuperSelective primer-based approach confirmed the presence of mutant DNA in patients with sequencing results demonstrating the presence of a mutation of interest in all instances, low copy numbers of mutant DNA were also detected among several patients with putative wild-type DNA only by sequencing, confirming the presence of heteroresistant M. tuberculosis below the threshold that can be amplified by standard methods of DNA sequencing.

Heteroresistance, known as the detection of both the mutant and the wild-type strains, is considered to be the early stage in the development of drug-resistant TB and is reflective of the slow evolution of bacteria from a sensitive to a resistant profile [36]. Heteroresistance was initially attributed to infections with multiple infecting M. tuberculosis strains (with mixed phenotypic resistance patterns); presumably containing strains from different lung regions that possess different drug susceptibilities. In recent years, the implementation of molecular diagnostic techniques has led to an alternate conceptualization of heteroresistance, recognizing that infection resulting from a single M. tuberculosis isolate may evolve to contain both mutant (drug-resistant) and wild-type (drug-susceptible) genomic DNA of the pathogen. Standard phenotypic culture-based drug resistance assays lead to the loss of these heteroresistant variants during the subculture process, interfering with an understanding of this phenomenon. The sensitivity of GenoType MTBDRplus and Line Probe Assay (LPA) Nipro for heteroresistance detection is about 5% [12,14]. Utilizing more advanced techniques, such as digital PCR, the detection threshold can reach up to 0.1% mutant: WT [10], though these assays require sophisticated equipment and/or are more expensive to implement in TB-endemic countries. By contrast, the method disclosed herein involves a carefully designed PCR primer strategy that can be used in widely available standard RT-PCR instruments. However, as with all other primer- and probe-based amplification assays, and unlike sequencing-based methods, it is necessary to know the exact nature of the polymorphism to develop a SuperSelective primer-based RT-PCR assay.

Early identification of heteroresistant TB may support efforts to optimize TB therapy on an individual basis, for example, with adherence to interventions or clinical decisions on drug selection, dosing, and therapeutic drug monitoring. SuperSelective primers, despite their extraordinary discriminatory ability (higher than conventional methods), are supremely easy to use. The only difference between these assays and conventional multiplex RT-PCR assays is the substitution of SuperSelective primers for conventional primers, while the sample preparation, amplification, and assessment are carried out in the same manner. Thus, RT-PCR assays with SuperSelective primers can be used as an accessory technique for resistance detection along with other methods of TB detection. Although the current study was performed using sputum samples from primary TB cases, the utility of SuperSelective PCR primers can be extended to re-treatment cases, which usually have a higher probability of a low to moderate number of copies of mutant DNA in the sputum samples, and they are relatively easier to detect.

Accordingly, the “SuperSelective” primer-based RT-PCR disclosed herein can be used to detect gene mutations in M. tuberculosis associated with heteroresistance in an abundant wild-type background. These assays can be used as diagnostic tools, which enable better and improved therapeutic management of drug-sensitive and drug-resistant TB cases in general and heteroresistant TB cases in particular.

### Methods and Uses for the Detection of Drug Resistance

The emergence of drug-resistant tuberculosis is a significant global health issue. The presence of heteroresistant Mycobacterium tuberculosis is critical to identifying fully drug-resistant tuberculosis cases. The currently available molecular techniques may detect one copy of mutant bacterial genomic DNA in the presence of about 1-1,000 copies of wild-type M. tuberculosis DNA.

To improve the limit of heteroresistance detection, the disclosure provides SuperSelective primer-based real-time PCR assays, which, by their unique assay design, enable selective and exponential amplification of selected point mutations in the presence of abundant wild-type DNA. SuperSelective primers are designed to detect genetic mutations associated with M. tuberculosis resistance to anti-tuberculosis drugs, such as isoniazid and rifampin.

As discussed herein, the efficiency of the assay in detecting heteroresistant M. tuberculosis strains was evaluated using genomic DNA isolated from laboratory strains and clinical isolates from the sputum of tuberculosis patients. The results showed that the assays detected heteroresistant mutations with a specificity of 100% in a background of up to 10,000 copies of wild-type M. tuberculosis genomic DNA, corresponding to a detection limit of 0.01%. Therefore, the SuperSelective primer-based RT-PCR assay is an ultrasensitive tool that can efficiently diagnose heteroresistant tuberculosis in clinical specimens and contributes to an enhanced understanding of drug-resistance mechanisms. This approach can improve the management of antimicrobial resistance in tuberculosis and other infectious diseases.

In one embodiment, the assay method includes amplifying a first nucleic acid target sequence in a reaction mixture with a first primer pair specific for a portion of a first region selected from the group consisting of a katG gene, an inhA promoter, and an rpoB gene to generate a first amplicon and detecting the first amplicon, whereby the presence of the first amplicon is indicative of the presence of drug resistance. The first forward primer or the first reverse primer or both can be a SuperSelective primer as disclosed herein. Any suitable primer-dependent amplification reaction can be used to carry out the amplifying step.

### 1. Primer-Dependent Amplification Reactions

Primer-dependent amplification reactions useful in methods of this invention may be any suitable exponential amplification method, including the polymerase chain reaction (PCR), either symmetric or non-symmetric, the ligase chain reaction (LCR), the nicking enzyme amplification reaction (NEAR), strand-displacement amplification (SDA), nucleic acid sequence-based amplification (NASBA), transcription-mediated amplification (TMA), and rolling circle amplification (RCA). Preferred methods utilize PCR. In non-symmetric PCR amplification methods, for example, asymmetric PCR, one primer, the limiting primer, is present in a limiting amount so as to be exhausted prior to completion of amplification, after which linear amplification occurs, using the remaining primer, the excess primer. A non-symmetric PCR method useful is LATE-PCR (see, for example, European Patent EP 1,468,114; and Pierce et al. (2005) Proc. Natl. Acad. Sci. USA 102:8609-8614). If a non-symmetric amplification method is used, the multi-part primer can be either the excess primer or the limiting primer. In one example, the multi-part primer is the excess primer. In another example, the multi-part primer is the limiting primer.

Preferred methods also include digital PCR (see, for example, Vogelstein and Kinzler (1999) Proc. Natl. Acad. Sci. USA 98:9236-9241), where it is desirable to detect a large number of amplicons from a single mutant template molecule that is present in reactions that contain abundant wild-type molecules.

If the amplification reaction utilizes an RNA-dependent DNA polymerase (an example being NASBA), the amplification reaction can be isothermal.

If the amplification reaction utilizes a DNA-dependent DNA polymerase (an example being PCR), an original sample may contain either DNA or RNA targets. For such amplifications, the intended target sequence primed by a multi-part primer according to this invention can be a DNA sequence that either occurs in an original sample or is made by reverse transcribing RNA sequences that occur in the original sample. If the multi-part primer is used for reverse transcription, the intended target sequence can be RNA or cDNA.

Primer-dependent amplification reactions can comprise repeated thermal cycles of primer annealing, primer extension, and strand denaturation (strand melting). Primer annealing may be performed at a temperature below the primer-extension temperature (for example, three-temperature PCR), or primer annealing and primer extension may be performed at the same temperature (for example, two-temperature PCR). The overall thermal profile of the reaction may include repetitions of a particular cycle, or temperatures/times may be varied during one or more cycles. For example, once amplification has begun and the priming sequence of a multi-part primer is lengthened, a higher annealing temperature appropriate for the longer primer might be used to complete the amplification reaction.

Assay methods described herein may include the detection of an amplified target sequence or an amplicon. The methods are not limited to particular detection schemes. Detection may be performed following amplification, as by gel electrophoresis. Alternately, homogeneous detection may be performed in a single tube, well, or other reaction vessel during (real time) or at the conclusion (end point) of the amplification reaction using reagents present during amplification. Alternatively, using a microfluidic device, amplified products can be moved to a chamber in which they contact one or more detection reagents or isolating reagents, such as immobilized capture probes. Detection reagents include double-stranded DNA binding dyes, for example, SYBR Green, and fluorescently or luminescently labeled hybridization probes that signal upon hybridization, for example, molecular beacon probes or ResonSense® probes, or probes that are cleaved during amplification, for example, 5′-nuclease (TaqMan®) probes.

Assays described herein may include screening assays looking for the presence of any target when one of multiple possible targets may be present. For such assays, a multi-part primer is used for each possible target, but detection need not identify which target is present. Therefore, SYBR Green dye can be used as the detection reagent, as can a dual-labeled hybridization probe that signals indiscriminately, as can a 5′ functional sequence on the primers that signals indiscriminately. Assays that employ multi-part primers according to this invention include amplification and detection, which may include quantitation, of two or more target sequences simultaneously in a single reaction tube, reaction well, or other reaction vessel, where one needs to identify which target or targets are present. The amplification and detection in a single reaction tube of two or more target sequences that do not have sequence homology and are located in different positions in a genome (for example the simultaneous detection of rare single-nucleotide polymorphisms located in different genes) may include for each different intended target sequence, a specific, uniquely colored, hybridization probe, such as a molecular beacon probe, a ResonSense® probe, or a 5′-nuclease (TaqMan®) probe that hybridizes to a unique sequence in either strand of the amplified product downstream from the multi-part primer. This applies not only to free-floating detector probes, but also to tethered probes such as molecular beacon probes.

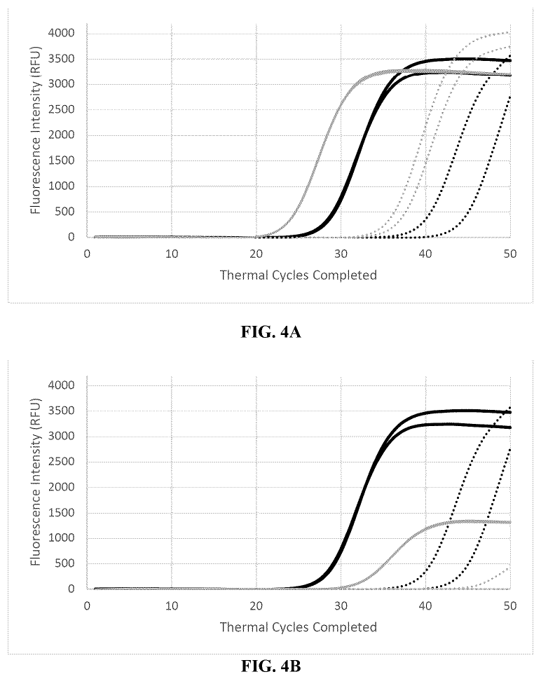

Alternatively, the multi-part primer for each different target sequence may include a labeled hairpin, such as that described in FIG. 4 of WO2014124290, the content of which is incorporated herein. In that case, two or more different multi-part primers, each specific for a different target sequence, and each labeled with a uniquely colored fluorescent label, can be used to simultaneously identify and quantitate each intended target sequence present in an individual sample.

### 2. Multiplex Assays

An attractive feature of SuperSelective primers disclosed herein is their potential use in multiplex assays that simultaneously measure the abundance of different mutant target sequences in the same clinical sample. The results of these assays can provide patient-specific information to tailor therapy for each individual.

An advantageous multiplex labeling strategy is based on the realization that, because there is no relation between the bridge sequence and the intended target sequence, assay designers are free to select a distinctly different bridge sequence for each of the different SuperSelective primers that are simultaneously present in a multiplex assay. Since the entire sequence of each primer becomes an integral part of the amplicon that is generated when that primer binds to its mutant target, the distinctive nucleic acid sequence of the bridge segment can serve as a “serial number” within that amplicon that identifies the mutant target from which it was generated.

These identifying bridge sequences can be relatively long (e.g., 20 nucleotides in length to assure their uniqueness), and the primers can be designed to form correspondingly short intervening sequences within the template. To simultaneously detect and quantitate different mutant target sequences that are present in a clinical sample, a set of specific molecular beacon probes (Tyagi et al., (1996) Nat. Biotechnol. 14, 303-308, Tyagi et al., (1998) Nat. Biotechnol., 16, 49-53, and Bonnet et al., (1999) Proc. Natl. Acad. Sci. USA, 96, 6171-6176) can be included in the real-time, gene amplification reactions, each specific for the complement of the distinctive bridge sequence of one of the SuperSelective primers, and each labeled with a differently colored fluorophore.

In these reactions, the concentration of the SuperSelective forward primers should be limited, and the linear reverse primers should be present in excess, thereby assuring that the reactions will not be symmetric, and that the molecular beacons will be able to bind to virtually all of the target amplicons that are synthesized in excess, without significant competition from less abundant complementary amplicons (Pierce et al., (2005) Proc. Natl. Acad. Sci. USA, 102, 8609-8614). These multiplex assays can even distinguish different mutations that occur in the same codon, since a SuperSelective primer designed to detect a particular mutation will discriminate against a neighboring or alternative mutation in the same way that it discriminates against a wild-type target sequence.

Another multiplex strategy is shown in FIG. 18 of WO2014124290, the content of which is incorporated herein. Where there is sequence homology between or among intended target sequences in a multiplex assay, a unique sequence can be introduced by utilizing for each different intended target sequence a unique bridge sequence. As explained above, the reverse primer copies the entire forward (multi-part) primer into the reverse product strand, so in subsequent cycles of amplification the entire multi-part primer (anchor sequence, bridge sequence, and foot sequence) is complementary to the product made by extension of the reverse primer. In multiplex assays, it is important that only one multi-part primer, the “correct” primer that was so copied, hybridizes to, and primes that reverse product strand. It will be appreciated that, therefore, one must make the bridge sequence of the “correct” multi-part primer sufficiently distinct to prevent another multi-part primer from priming that reverse product strand (so-called “cross hybridization”). That having been done, a specific, uniquely colored hybridization probe, free-floating or tethered to the primer, that is targeted against the complement of the bridge sequence will signal amplification of only one intended target and will not signal falsely by hybridizing to the multi-part primer itself. Similarly, only the “correct” multi-part primer with a uniquely colored hairpin tail will hybridize to the reverse product strand and signal.

### Compositions and Kits

This disclosure encompasses a composition or reaction mixture comprising the aforementioned primers and reagents for carrying out the methods described above. For example, the composition can comprise one or more reagents selected from the group consisting of a nucleic acid polymerase, deoxyribonucleoside triphosphates, and a detecting agent.

The detecting agent can be an oligonucleotide probe, such as a molecular beacon probe or a Yin-Yang probe that is labeled with a fluorophore and a quencher. See e.g., U.S. Pat. Nos. 5,925,517, 6,103,476, 6,150,097, 6,270,967, 6,326,145, and 7,799,522. The composition can also comprise, in addition to the above reagents, one or more of: a salt, e.g., NaCl, MgCl₂, KCl, or MgSO₄; a buffering agent, e.g., a Tris buffer, N-(2-Hydroxyethyl)-piperazine-N′-(2-ethanesulfonic acid) (HEPES), 2-(N-Morpholino) ethanesulfonic acid (MES), MES sodium salt, 3-(N-Morpholino) propanesulfonic acid (MOPS), N-tris-(hydroxymethyl)-methylaminopropanesulfonic acid (TAPS); a solubilizing agent; a detergent, e.g., a non-ionic detergent such as Tween-20; a nuclease inhibitor; and the like.

The reaction components used in an amplification and/or detection process may be provided in a variety of forms. For example, the components (e.g., enzymes, deoxyribonucleoside triphosphates, adaptors, blockers, and/or primers) can be suspended in an aqueous solution or as a freeze-dried or lyophilized powder, pellet, or bead. In the latter case, the components, when reconstituted, form a complete mixture of components for use in an assay.

The above-described reaction mixture can further include an effective amount of a selectivity-enhancing reagent, such as a Hofmeister salt. Examples include tetramethylammonium chloride (TMAC) and bis-tetramethylammonium oxalate.

This disclosure further includes reagent kits containing reagents for performing the above-described amplification methods, including amplification and detection methods. To that end, one or more of the reaction components for the methods disclosed herein can be supplied in the form of a kit for use in the detection of a target nucleic acid. In such a kit, an appropriate amount of one or more reaction components is provided in one or more containers or bound to a substrate (e.g., by electrostatic interactions or covalent bonding).

The kit described herein includes one or more of the primers described above. The kit can include one or more containers containing one or more primers of the invention. A kit can contain a single primer in a single container, multiple containers containing the same primer, a single container containing two or more different primers of the invention, or multiple containers containing different primers or containing mixtures of two or more primers. Any combination and permutation of primers and containers is encompassed by the kits of the disclosure.

The kit can also contain additional materials for practicing the above-described methods. In some embodiments, the kit contains some or all of the reagents, and materials for performing a method that uses a primer according to the disclosure. The kit thus may comprise some or all of the reagents for performing a PCR reaction using the primer disclosed herein. Some or all of the components of the kits can be provided in containers separate from the container(s) containing the primer. Examples of additional components of the kits include, but are not limited to, one or more different polymerases, one or more primers that are specific for a control nucleic acid or for a target nucleic acid, one or more probes that are specific for a control nucleic acid or for a target nucleic acid, buffers for polymerization reactions (in 1× or concentrated forms), and one or more dyes or fluorescent molecules for detecting polymerization products. The kit may also include one or more of the following components: supports, terminating, modifying or digestion reagents, osmolytes, and an apparatus for detecting a detection probe.

The reaction components used in an amplification and/or detection process may be provided in a variety of forms. For example, the components (e.g., enzymes, nucleotide triphosphates, probes and/or primers) can be suspended in an aqueous solution or as a freeze-dried or lyophilized powder, pellet, or bead. In the latter case, the components, when reconstituted, form a complete mixture of components for use in an assay.

A kit or system may contain, in an amount sufficient for at least one assay, any combination of the components described herein, and may further include instructions recorded in a tangible form for use of the components. In some applications, one or more reaction components may be provided in pre-measured single use amounts in individual, typically disposable, tubes or equivalent containers. With such an arrangement, the sample to be tested for the presence of a target nucleic acid can be added to the individual tubes and amplification carried out directly. The amount of a component supplied in the kit can be any appropriate amount, and may depend on the target market to which the product is directed. General guidelines for determining appropriate amounts may be found in, for example, Joseph Sambrook and David W. Russell, Molecular Cloning: A Laboratory Manual, 3rd edition, Cold Spring Harbor Laboratory Press, 2001; and Frederick M. Ausubel, Current Protocols in Molecular Biology, John Wiley & Sons, 2003.

The kits of the disclosure can comprise any number of additional reagents or substances that are useful for practicing a method of the invention. Such substances include but are not limited to reagents (including buffers) for the lysis of cells, divalent cation chelating agents or other agents that inhibit unwanted nucleases, control DNA for use in ensuring that primers, the polymerase, and other components of reactions are functioning properly, DNA fragmenting reagents (including buffers), amplification reaction reagents (including buffers), and wash solutions. The kits of the invention can be provided at any temperature. For example, for storage of kits containing protein components or complexes thereof in a liquid, it is preferred that they are provided and maintained below 0° C., preferably at or below −20° C., or otherwise in a frozen state.

The container(s) in which the components are supplied can be any conventional container that is capable of holding the supplied form, for instance, microfuge tubes, ampoules, bottles, or integral testing devices, such as fluidic devices, cartridges, lateral flow, or other similar devices. The kits can include either labeled or unlabeled nucleic acid probes for use in amplification or detection of target nucleic acids. In some embodiments, the kits can further include instructions to use the components in any of the methods described herein, e.g., a method using a crude matrix without nucleic acid extraction and/or purification.

The kits can also include packaging materials for holding the container or a combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like).

### EXAMPLES

### Example 1

This example descibes the material and methods used in Example 2 below.

### 1.1. Selected Antibiotic-Drug-Resistant Mutations

SuperSelective primers were designed to amplify mutant M. tuberculosis DNA corresponding to INH and RIF resistance in a WT background. For INH resistance, SuperSelective primers were designed to target katG (S315T, AGC→ACA, and AGC→ACC) and the promoter region of inhA (−8T→A, −15C→T, and −17G→T) [25-27]. For RIF resistance, the primers were designed to focus on the most common mutations in the RIF-resistance-determining region (RRDR) of rpoB (D516V, GAC→GTC; H526D, CAC→GAC; H526Y, CAC→TAC; and S531L, TCG→TTG) [37].

### 1.2. Structure and Functionality of SuperSelective Primers

The SuperSelective PCR primers were designed to have a minimal probability of initiating synthesis on wild-type sequences, even when the only difference between the mutant and the WT targets was a single-nucleotide polymorphism. The SuperSelective primer designs were adapted from previous reports [25,38]. Briefly, SuperSelective primers used in the present study consist of the three sequence segments in order from 5′ to 3′:
(i) an anchor sequence complementary to the target DNA that is sufficiently long to ensure strong hybridization to its DNA target fragment under PCR annealing conditions;
(ii) a distinctive bridge sequence, which is non-complementary to the corresponding intervening sequence in the DNA target fragment; and (iii) a short 3;-foot sequence that is entirely complementary to the corresponding sequence in the mutant DNA target fragments, but is discrepant to the corresponding sequence in the related WT DNA fragments.

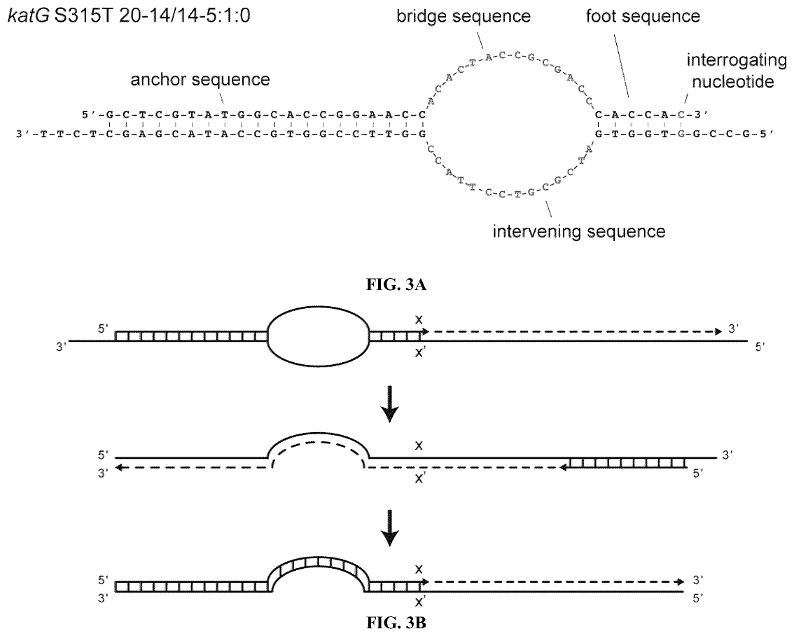

In a PCR assay, the function of a SuperSelective primer is to maintain a delicate balance between the anchor sequence (long 5′ segment), which promotes its efficient binding to a gene of interest, and the foot sequence (short 3′ segment), which restricts binding to the subsequence that includes the target mutation. Complementarity between the “interrogating nucleotide” in the foot to the corresponding nucleotide in the mutant target sequence makes the foot and mutant target perfectly complementary and leads to priming of the synthesis of an amplicon, while the mismatch at the corresponding nucleotide in the WT target sequence inhibits the amplification of the WT target. DNA-DNA hybrids formed by the anchor sequence with target DNA and the short foot sequence with target DNA are separated from each other by a single-stranded bubble formed by the non-complimentary bridge sequence (which should not form any secondary structures) and the intervening sequence in the DNA target fragment. This bubble effectively separates the primer's polymerization initiation function from the primer's mutant target recognition function. FIG. 3A shows an example of a SuperSelective primer bound to its mutant target sequence. This particular primer was designed to selectively amplify DNA fragments containing a katG S315T (AGC→ACA) single-nucleotide polymorphism, which is commonly associated with INH resistance in TB patients.

The nomenclature for primer labeling was as described earlier [25]. For example, a SuperSelective primer 20-14/14-5:1:0 (shown in FIG. 3A) indicates that the anchor sequence is 20 nucleotides long, the bridge sequence is 14 nucleotides long (across from an intervening sequence in the template that is 14 nucleotides long), and the foot sequence is 6 nucleotides long, with one interrogating nucleotide located at the last position from the primer's 3′ end.

In an RT-PCR assay with a SuperSelective primer, the selective step occurs when a SuperSelective primer (designed against the DNA (−) strand template) is bound to the original DNA sample being analyzed. The foot sequence of the SuperSelective primer then initiates the synthesis of an amplicon, and as the reaction proceeds, the entire sequence of the Super-Selective primer (including the “artificial” bridge sequence) is incorporated into that (+) amplicon. In the successive thermal cycles, the resulting amplicons are amplified efficiently in the normal manner, with the entire SuperSelective primer sequence serving as a long conventional primer that is completely complementary to the (−) amplicon, which includes the complement of the primer's bridge sequence in place of the intervening sequence that was present in the original template (FIG. 3B).

### 1.3. Primers, Molecular Beacons, and POR Reagents

In all assays, a SuperSelective forward primer was paired with a conventional reverse primer unless mentioned otherwise. SuperSelective primer sequences were assessed using the Mfold web server and the OligoAnalyzer computer program (Integrated DNA Technologies, Coralville, IA, USA) to ensure that these sequences are unlikely to form internal secondary structures such as hairpin loops, self-dimers, or heterodimers with conventional reverse primers. Conventional and SuperSelective primers were purchased from Integrated DNA Technologies. Molecular beacon probes were purchased from LGC Biosearch Technologies (Petaluma, CA, USA). Platinum Taq DNA polymerase, deoxynucleotide triphosphates (dNTPs), and SYBR Green were purchased from Thermo Fisher Scientific, Inc. (Waltham, MA, USA). Nuclease-free water was purchased from Ambion, Inc. (Austin, TX, USA).

### 1.4. Target DNA

Complete WT and mutant (AGC→ACA) katG genes of M. tuberculosis were cloned into the vector pcDNA3.1 and used as the source of mutant plasmid DNA (pDNA) for assays. For the optimization of assays to detect AGC→ACC mutation, plasmids (pUC) containing a 584 bp subsequence of the katG gene (WT and AGC→ACC) were synthesized (Integrated DNA Technologies). To detect inhA promoter mutations, plasmids (pUC) containing a 387 base pair region covering mutations (−8T→A, −15C→T, and −17G→T) and the corresponding WT were synthesized (Integrated DNA Technologies). To detect rpoB mutations, a 488 base pair subsequence of mutant genes containing mutations D516V (GAC→GTC), rpoB H526D (CAC→GAC), H526Y (CAC→TAC), rpoB S531L (TCG→TTG) and the corresponding WT were synthesized (Integrated DNA Technologies). The selected M. tuberculosis genes targeting INH and RIF resistance are commonly associated with MDR-TB cases [37,41-43].

The plasmids containing mutations of interest were linearized by restriction digestion for 120 min at 37° C. in 50 μL containing 10 units of restriction endonuclease ClaI with appropriate buffer supplied by the manufacturer (New England Biolabs, Ipswich, MA, USA). The reaction mix was incubated for 20 min at 65° C. to inactivate the enzyme, and the concentration of each linearized plasmid was determined in a NanoDrop spectrophotometer (Thermo Fisher Scientific, Inc., Waltham, MA, USA). The plasmids were diluted in nuclease-free water to create stock solutions containing known quantities of linearized target plasmids/μL.

The genomic DNA (gDNA) of INH- and RIF-resistant M. tuberculosis was provided by the Kreiswirth laboratory (CDI, Hackensack Meridian Health, Nutley, NJ, USA). The gDNA sequences were verified by DNA sequence analysis (Integrated DNA Technologies). Wild-type H37Rv DNA was obtained from BEI resources (NIAID, NIH, Washington, DC, USA).

### 1.5. Preparation of DNA Samples

Template DNA for RT-PCR assays was prepared using pDNA or gDNA. A calculated number of mutant DNA copies was mixed with a calculated number of WT DNA copies to create varying ratios of mutant DNA against a WT background. Serial dilutions of mutant DNA (10⁵-1 copies) were mixed with 10⁵ or 10⁴ copies of WT DNA. Tested mutant WT ratios were 10⁵:10⁵, 10⁴:10⁵, 10³:10⁵, 10²:10⁵, 50:10⁵, 25:10⁵, 10:10⁵, 1:10⁵, 0:10⁵ and 10⁵:10⁴, 10⁴:10⁴, 10³:10⁴, 10²:10⁴, 50:10⁴, 25:10⁴, 10:10⁴, 1:10⁴, 0:10⁴. Mutant DNA copies 10⁵, 10⁴, 10³, 10², 50, 25, 10 and 1 were used as controls.

### 1.6. Assay Composition and Testing Procedure

Monoplex RT-PCR assays were performed with a SuperSelective forward primer and a conventional reverse primer on an AriaMx Real-Time PCR System (Agilent Technologies, Santa Clara, CA, USA). All amplifications were carried out in 0.2 mL clear tubes (Agilent Technologies, Santa Clara, CA, USA) in a final volume of 25 μL, containing 1× PCR buffer supplemented with 25 mM tetramethylammonium chloride (TMAC) (Sigma-Aldrich, St. Louis, MO, USA), 0.25% Tween20 (Sigma-Aldrich, St. Louis, MO, USA), 3 mM MgCl₂, 0.05 U/μL Platinum Taq DNA polymerase (Thermo Fisher Scientific, Inc., Waltham, MA, USA), 0.25 mM dNTP mix, 0.1 μM each of the forward and the reverse primer, and 0.5× SYBR Green (Thermo Fisher Scientific, Inc., Waltham, MA, USA) to monitor template amplification. The reaction mixtures were incubated for 2 min at 95° C. to activate the Platinum Taq DNA polymerase, followed by 50-65 cycles of DNA denaturation at 95° C. for 15 s, primer annealing at 60° C. for 20 s, and extension at 72° C. for 20 s. Fluorescence was monitored and measured during the annealing step.

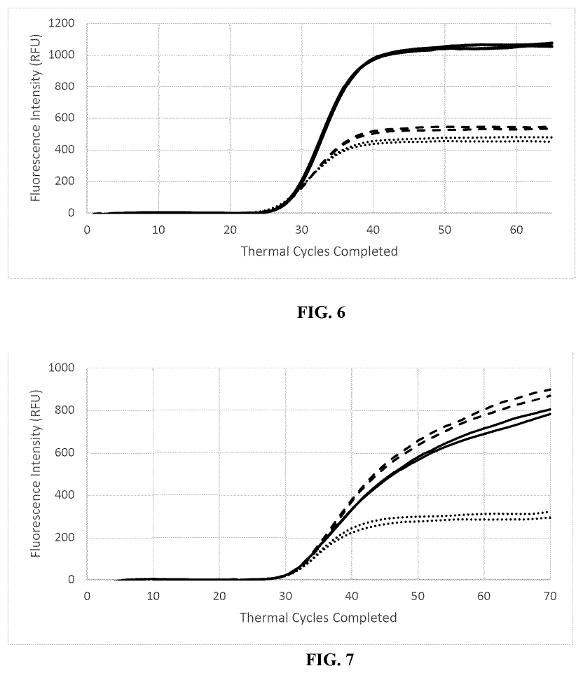

For the detection of mutants using molecular beacon probes in monoplex assays, asymmetric PCR conditions were optimized (FIGS. 6 and 7) to maximize the intensity of fluorescence signals. RT-PCR assay conditions were the same as described above, except that the forward and reverse primers were used at a 1:5 ratio, with final concentrations of 0.1 μM and 0.5 μM, respectively. This assay used 0.1 μM of molecular beacon probe instead of SYBR Green to monitor amplification.

Multiplex assays with molecular beacon probes were carried out in 0.2 mL white polypropylene PCR tubes (USA Scientific, Ocala, FL, USA) in a Bio-Rad CFX96 Real-Time System (Bio-Rad Laboratories, Hercules, CA, USA). RT-PCR assay conditions were the same as those used in the monoplex assays, except that three primer pairs and three probes were used (katG, inhA, rpoB) for multiplexing. The final concentration of each forward primer (katG, inhA, rpoB) was 0.1 μM, each reverse primer (katG, inhA, rpoB) was 0.5 μM, probe katG was 0.25 μM, probe inhA was 0.1 μM, and probe rpoB was 0.25 μM.

The RT-PCR assays were initially optimized on linearized plasmids containing a subsequence of the gene containing respective mutations as template DNA because of the availability of a large amount of material. The assay results were then confirmed by testing the optimized assay conditions on M. tuberculosis genomic DNA. RT-PCR conditions were tested for mutant DNA template at copy numbers 10⁵ to 1 (i.e., 10⁵, 10⁴, 10³, 10², 10¹, and 10⁰) in a background of 10⁵ or 10⁴ copies of the WT DNA template. Mutant DNA at the copy numbers 10⁵ to 1 with no background of WT DNA was used as an assay control. Nuclease-free water was used as a no-template control. Experiments were performed on duplicate samples and repeated at least three times.

### 1.7. Clinical Samples

Twenty-three M. tuberculosis DNA samples were isolated from archived sputum samples of anonymized TB patients with unknown drug resistance. These patient-health information (PHI) de-identified and anonymized sputum specimens were collected as part of routine TB diagnosis screening by conventional methods, including bacterial culture and smear microscopy. Hence, no informed consent was required to extract bacterial DNA from the specimen, and the archived samples were accessed exclusively to rationally generate beneficial and scientifically valid assays to improve patient health. To isolate the gDNA, the sputum samples were first decontaminated and homogenized by using N-acetyl-1-cystine-sodium (NALC-NaOH), then 2 mL of sputum was taken in an Oak Ridge tube, and an equal amount of 1% N-acetyl-L-cysteine, 4% sodium hydroxide, and 2.9% sodium citrate was added. The suspension was incubated at 37° C. for 15 min and added to 45 mL of phosphate buffer, followed by centrifugation at 5,000 rpm for 15 min. The obtained pellet was re-suspended in 1 mL of nuclease-free water. For DNA extraction, 1 mL of the decontaminated sample was spun again and re-suspended in 50 μL of Genolyse Lysis buffer (Hain Lifesciences GmbH, Hardwiesentrabe, Nehren, Germany), followed by heat-killing at 95° C. for 5 min to lyse the bacterial cells. Subsequently, Genolyse Neutralizing buffer (50 μL) was added to the suspension, and the resulting DNA in the supernatant was pelleted by centrifugation at 13,000 rpm for 5 min.

### Example 2

### 2.1. SuperSelective Primer Design

The inventors first designed a prototype SuperSelective primer to detect inhA promoter mutation −15C→T and optimized the primer configuration and PCR conditions to achieve maximum sensitivity for mutant detection. A SuperSelective primer 18-14/14-6:1:0 (inh15_SSP3) was paired with a conventional reverse primer and tested in an RT-PCR assay to detect one copy of −15C→T mutant in a background of 10⁵ copies of WT DNA. Linearized pUC plasmids containing a cloned sequence carrying the inhA promoter mutation −15C→T was used as a template for these experiments, and its WT counterpart was used as background. The results showed that the primer design efficiently amplified as low as one copy of mutant DNA (in three out of six replicates) in a background of 10⁵ copies of WT DNA. Amplification of the mutant in the control set of the experiment (10⁵ copies of mutant DNA without the WT background) down to one copy confirmed that the amplification signal for the mutant from low copy numbers was specific and not a product of the non-specific binding of primers to the background WT pDNA. Although this SuperSelective primer design also resulted in the amplification of WT DNA (one out of six replicates), the WT amplification peaks were seen occasionally at PCR cycle (Ct)>40, which overlapped with the amplification peaks with low copy numbers (ten and one) of mutant. The non-suppression of the unintended WT target amplification using this SuperSelective primer could be attributed to the high GC content in M. tuberculosis gDNA [30], leading to a firm binding of the anchor and/or foot regions to the WT template.

The main objective of this investigation was to design primers adequate for early amplification of the mutant (ideally Ct 19-25 for 10⁵ copies) to attain amplification down to 1 copy within a short number of RT-PCR cycles (ideally 50 cycles), with complete suppression of WT background. Therefore, to determine optimal parameters for this assay, the inventors tweaked the SuperSelective primer design in different parts of the PCR (owing to different functions), making one to two variations at a time, and tested these modified designs under different annealing temperatures (Tm) and assay conditions (Table 1).

TABLE 1
RT-PCR assay conditions tested for the optimization of SuperSelective
primers for the detection of inhA promoter mutation −15C→T.

| | | Annealing | TMAC |
|---|---|---|---|
| Forward Primer | Reverse Primer | Temperature (° C.) | (mM)* |
| inh15_SSP2 (18-14/21-6:1:0) | inhA_rev1 | 60 | 25 |
| inh15_SSP3 (18-14/14-6:1:0) | inhA_rev1 | 60, 62 | 25 |
| inh15_SSP3 (18-14/14-6:1:0) | inhA_rev2 | 60 | 25 |
| inh15_SSP4 (18-14/13-7:1:0) | inhA_rev1 | 60, 62, 66 | 25 |
| inh15_SSP4 (18-14/13-7:1:0) | inhA_rev3, inhA_rev4 | 60 | 25 |
| inh15_SSP5 (18-15/13-7:1:0) | inhA_rev1 | 60, 62 | 25 |
| inh15_SSP6 (18-17/13-7:1:0) | | | |
| inh15_SSP7 (18-20/8-7:1:0) | inhA_rev1 | 60, 64, 66 | 25 |
| inh15_SSP8 (18-8/8-7:1:0) | | | |
| inh15_SSP9 (18-8/20-7:1:0) | | | |
| inhA_for2 | SSP10 (18-14/14-6:1:0) | 60 | 25 |
| inh15_SSP4 (18-14/13-7:1:0) | | | |
| inh15_SSP4 (18-14/13-7:1:0) | SSP10 (18-14/14-6:1:0) | 60, 64 | 0, 25 |
| inh15_SSP7 (18-20/8-7:1:0) | | | |
| inhA_for2 | SSP11 (18-14/13-7:1:0) | 60 | 0, 25 |
| inhA_for4 | | | |
| inh15_SSP4 (18-14/13-7:1:0) | | | |
| inh15_SSP7 (18-20/8-7:1:0) | | | |
| inhA_for2 | SSP12 (18-14/8-6:1:0) | 60 | 0 |
| inhA_for4 | SSP13 (18-14/8-7:1:0) | | |
| inh15_SSP4 (18-14/13-7:1:0) | | | |
| inh15_SSP7 (18-20/8-7:1:0) | | | |
| inh15_SSP4 (18-14/13-7:1:0) | SSP10 (18-14/14-6:1:0) | 64, 66 | 0 |
| inh15_SSP7 (18-20/8-7:1:0) | SSP11 (18-14/13-7:1:0) | | |
| | SSP12 (18-14/8-6:1:0) | | |
| | SSP13 (18-14/8-7:1:0) | | |
| inh15_SSP4 (18-14/13-7:1:0) | SSP17, 18 | 60 | 25 |
| inh15_SSP22 | SSP10 (18-14/14-6:1:0) | 60 | 0 |
| inh15_SSP14-16, 19-21 | inhA_rev1 | 60 | 25 |
| inh15_SSP23-25 | SSP10 (18-14/14-6:1:0) | 60 | 25 |
| inh15_SSP3 (18-14/14-6:1:0) | inhA_rev1 | 60 | 0 |
| inh15_SSP4 (18-14/13-7:1:0) | | | |
| inh15_SSP7 (18-20/8-7:1:0) | | | |

*The addition of TMAC was always accompanied by inclusion of Tween 20 (0.25%)

### 2.2. Standardization of Primer Foot Length

To strengthen the binding of SuperSelective primers with the mutant DNA template, the inventors tested SuperSelective primer 18-14/13-7:1:0 (inh15_SSP4) with a longer foot length (8 nucleotides instead of 7 nucleotides) in the RT-PCR assay described as above. The resulting Ct values of 10⁵ copies of mutant and 10⁵ copies of WT DNA with this longer foot primer were plotted against the Ct values obtained with primer 18-14/14-6:1:0 (inh15_SSP3). The results showed that the extended foot length (8 nucleotides) in primer inh15_SSP4 helped in the early amplification of mutant DNA template (3-4 cycles early), but also enhanced the binding of SuperSelective primers with WT DNA (FIG. 4A). These results demonstrate that a shorter foot length may delay achieving the threshold cycle, leading to enhanced selectivity, whereas a longer foot length may reduce the selectivity of SuperSelective primers. From a thermodynamic standpoint, the improved selectivity with shorter foot lengths can be due to the higher ratio of the equilibrium abundance of perfectly complementary mutant foot hybrids compared to the equilibrium abundance of mismatched WT foot hybrids [25].

### 2.3. Standardization of the Size and Symmetry of the Bubble

In order to inhibit the amplification of the WT gene copy or to delay the amplification beyond 50 cycles, which would avoid overlap with the amplification of the mutant, the inventors tested SuperSelective primers 18-14/21-6:1:0 (inh15_SSP2) with increased lengths of the intervening sequence (from 14 to 21 nucleotides) (forming an asymmetric bubble) in RT-PCR assays. The resulting Ct values of 10⁵ copies of mutant and 10⁵ copies of WT with this primer were plotted against the Ct values obtained with 18-14/14-6:1:0 (inh15_SSP3), which has a shorter length of the intervening sequence (equal to the length of the bridge, forming a symmetric bubble) (FIG. 4B). Two reactions were carried out with each of the primers, of which 18-14/21-6:1:0 initiated the amplification of WT in one reaction and completely suppressed the amplification in the other sample. By contrast, the 18-14/14-6:1:0 primer amplified the targets of both WT replicate reactions. The asymmetric bubble with 18-14/21-6:1:0 also resulted in a Ct delay (˜5 cycles) in the amplification of the mutant target and a lower fluorescence intensity signal. These results signify that the asymmetricity of the bubble, created by the intervening sequence, may significantly affect the binding efficiency of a SuperSelective primer with its target and, thus, the amplification threshold, resulting in the formation of fewer amplicons.

The inventors also tested SuperSelective primers with a variation (15/17 nucleotides) in bridge length of the primer design 18-14/13-7:1:0 (inh15_SSP4). In all other aspects, the composition of the primers was the same, i.e., 18-15/13-7:1:0 (inh15_SSP5) and 18-17/13-7:1:0 (inh15_SSP6). No significant difference in Ct values was found for 10⁵ copies of mutant, or 10⁵ copies of WT obtained with primers inh15_SSP4 and inh15_SSP5, which have bridge lengths of 14 and 15 base pairs, respectively. Primer inh15_SSP6 with a bridge length of 17 base pairs formed a bigger and more asymmetric bubble, delaying the amplification of 10⁵ copies of the mutant or 10⁵ copies of WT by 1 cycle. The window of discrimination (ΔCt) between the Ct for the mutant and the WT was similar for these two primers.

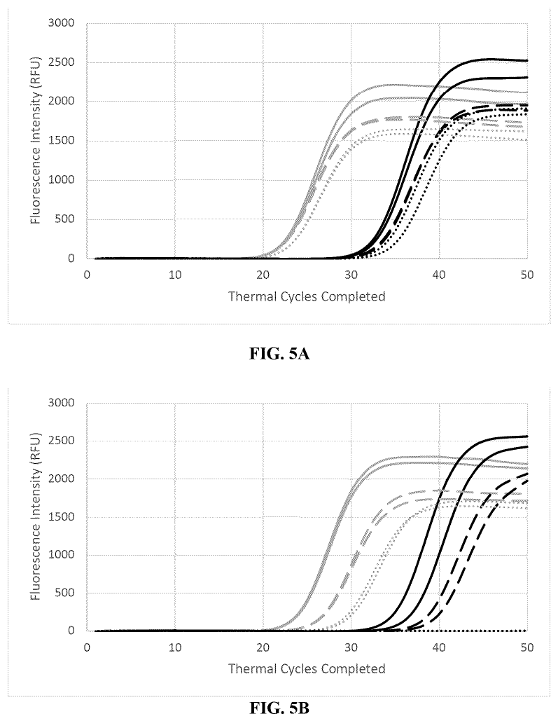

Furthermore, the inventors tested different bridge-intervening sequence length combinations—20/8, 8/8, and 8/20 (inh15_SSP7-9)—in the SuperSelective primer designs. SuperSelective primers with a long bridge but short intervening sequence 18-20/8-7:1:0 (SSP7), short bridge-short intervening sequence 18-8/8-7:1:0 (SSP8) and short bridge-long intervening sequence 18-8/20-7:1:0 (SSP9) were tested at Tm 60-66° C., while all other RT-PCR conditions were maintained similar to those above (Table 1). Amplification of the mutant target using the SuperSelective primer with a symmetric bubble 18-8/8-7:1:0 (SSP8) was the least affected by changes in Tm, although the window of discrimination (ΔCt) between the Ct for the mutant and the Ct for the WT increased with increasing temperature (FIG. 5A). Amplification of the mutant target using both SuperSelective primers forming an asymmetric bubble (18-20/8-7:1:0 and 18-8/20-7:1:0) was delayed according to the Tm of the assay. Only 18-20/8-7:1:0 suppressed the amplification of the WT pDNA template (at Tm 66° C.), but also caused a delay (5 cycles) in the amplification of the intended mutant target (FIG. 5B).

### 2.4. Changes in Foot Design

In some of the potential SuperSelective primer designs tested previously, the inventors introduced a mismatch in the foot sequence at the fourth position from the interrogating nucleotide, i.e., 18-14/14-6:1:0 (inh15_SSP14), 18-14/13-7:1:0 (inh15_SSP15), and 18-20/8-7:1:0 (inh15_SSP14-inh15_SSP16) to destabilize the binding of the foot sequence with the WT DNA. The inventors also tested SuperSelective primers 18-14/14-3:2:1, 18-14/14-6:2:1, and 18-14/14-6:2:0 (inh15_SSP23 to inh15_25, respectively) by introducing a mismatch nucleotide immediately after the interrogating nucleotide. In theory, this design would weaken the binding of the foot sequence with its target, with a more pronounced effect on the WT target with two mismatches compared to the mutant with a single mismatch. The inventors also investigated the effect of varying the location of the interrogating nucleotide in the foot sequence on the primer's ability to discriminate mutant templates from WT templates using primer designs 18-14/14-6:1:2, 18-14/13-7:1:2, 18-20/8-7:1:2, and 18-14/14-3:2:1 (inh15_SSP19 to inh15_SSP22, respectively). The results from these PCR assays indicate that the variations in foot sequence design aimed to prevent the amplification of WT DNA led to a 8-16 cycle delay in the amplification of the mutant target.

### 2.5 SuperSelective Reverse Primers

To decrease the binding of the SuperSelective forward primers paired with conventional reverse primers to WT DNA, the SuperSelective forward primers were paired with SuperSelective reverse primers [31]. The inventors designed SuperSelective reverse primers 18-14/14-6:1:0 (inh15_SSP10) and 18-14/13-7:1:0 (inh15_SSP11) with varying foot length (7/8 nucleotides), 18-14/8-6:1:0 (inh15_SSP12) and 18-14/8-7:1:0 (inh15_SSP13) with varying foot length (7/8 nucleotides plus a shorter intervening sequence (8 nucleotides), and 20-14/14-6:1:0 (inh15_SSP17) and Rev 20-14/13-7:1:0 (inh15_SSP18) with varying foot length (7/8 nucleotides) and a longer anchor (20 nucleotides). These primers were paired with SuperSelective primers 18-14/13-7:1:0 (inh15_SSP4) and 18-20/8-7:1:0 (inh15_SSP7) or their respective anchor sequences, which acted as conventional forward primers. The effect of foot length and intervening sequence on a reverse SuperSelective primer paired with a conventional forward primer was as shown above for a forward SuperSelective primer paired with a conventional reverse primer. The inventors compared the Ct values for detecting 10⁵ copies of mutant and 10⁵ copies of WT with each of these SuperSelective primers paired either with a conventional second primer to their pairing with another SuperSelective primer. The results showed that using two SuperSelective primers drastically increased the window of discrimination (ΔCt) for mutant and WT targets from <5 to 8-13, yet it did not completely suppress the amplification of WT templates.

Some of these variations in SuperSelective primer design were also tested on other mutations in the panel, i.e., inhA (−8T→A, −17G→T), katG (S315T, AGC→ACA, and AGC→ACC), and rpoB (D516V, GAC→GTC; H526D, CAC→GAC; H526Y, CAC→TAC; and S531L, TCG→TTG) (Table 2). All of the tested SuperSelective primer designs and combinations amplified the unintended WT target, or the suppression of WT DNA was accompanied by a delay in the amplification of 10⁵ copies of mutant and/or suppression of amplification of ten and one copy of mutant. The inventors repeatedly observed an overlap of low copy number mutant (ten and one) and WT amplification peaks. It should be noted that the SYBR Green reagent used to monitor amplification in these RT-PCR assays is a DNA-intercalating dye that detects specific and non-specific amplification products. Therefore, to confirm the specificity of the amplified products tested by SYBR Green, a subset of SuperSelective primers was tested for amplification using molecular beacon probes.

TABLE 2
List of primers used for katG, inhA and rpoB mutations.

| | | | Strand | | |
|---|---|---|---|---|---|
| Gene | Mutation | Primer ID | +/- | Design | Sequence |
| katG | S315T | katG315T2_ | + | 20-14/14-5:2:0 | GCTCGTATGGCACCGGAAC |
| | AGC→ACA | SSP1 | | | CACACTACCGCGACCCACC |
| | | | | | ACA (SEQ ID NO: 7) |
| katG | S315T | katG315T1_ | + | 20-14/14-5:1:0 | GCTCGTATGGCACCGGAAC |
| | AGC→ACC | SSP1 | | | CACACTACCGCGACCCACC |
| | | | | | AC (SEQ ID NO: 8) |
| katG | S315T | katG315T1_ | + | 20-14/13-6:1:0 | GCTCGTATGGCACCGGAAC |
| | AGC→ACC | SSP2 | | | CTCACTACAGAAACCTCACC |
| | | | | | AC (SEQ ID NO: 9) |
| katG | | katG_rev1 | − | | ATTTCGTCGGGGTGTTCGT |
| | | | | | C (SEQ ID NO: 10) |
| katG | | katG_rev2 | − | | TCGTAGCCGTACAGGATCT |
| | | | | | CG (SEQ ID NO: 11) |
| inhA | −8T→A | inh8_SSP1 | − | 18-14/14-6:1:0 | GTTTGGCCCCTTCAGTGGAA |
| | | | | | TATTGTTGATAACCGACAT |
| | | | | | (SEQ ID NO: 12) |
| inhA | −8T→A | inh8_SSP2 | − | 20-14/14-6:1:0 | GGGTTTGGCCCCTTCAGTG |
| | | | | | GAATATTGTTGATAACCGAC |
| | | | | | AT (SEQ ID NO: 13) |
| inhA | −15C→T | inh15_SSP2 | − | 18-14/21-6:1:0 | GTTTGGCCCCTTCAGTGGAA |
| | | | | | TATTGTCATGTTCCTATCA |
| | | | | | (SEQ ID NO: 14) |
| inhA | −15C→T | inh15_SSP3 | − | 18-14/14-6:1:0 | CCCTTCAGTGGCTGTGGCTA |
| | | | | | TATTGTCATGTTCCTATCA |
| | | | | | (SEQ ID NO: 15) |
| inhA | −15C→T | inh15_SSP4 | − | 18-14/13-7:1:0 | CCCTTCAGTGGCTGTGGCTA |
| | | | | | TATTGTCATGTTACCTATCA |
| | | | | | (SEQ ID NO: 16) |
| inhA | −15C→T | inh15_SSP5 | − | 18-15/13-7:1:0 | CCCTTCAGTGGCTGTGGCTA |
| | | | | | TATTAGTCATGTTACCTATC |
| | | | | | A (SEQ ID NO: 17) |
| inhA | −15C→T | inh15_SSP6 | − | 18-17/13-7:1:0 | CCCTTCAGTGGCTGTGGCTA |
| | | | | | TATTATCGTCATGTTACCTAT |
| | | | | | CA (SEQ ID NO: 18) |
| inhA | −15C→T | inh15_SSP7 | − | 18-20/8-7:1:0 | CAGTGGCTGTGGCAGTCAT |
| | | | | | CTAGTACACTCGTCATGTTAC |
| | | | | | CTATCA (SEQ ID NO: 19) |
| inhA | −15C→T | inh15_SSP8 | − | 18-8/8-7:1:0 | CAGTGGCTGTGGCAGTCAG |
| | | | | | TCATGTTACCTATCA (SEQ |
| | | | | | ID NO: 20) |
| inhA | −15C→T | inh15_SSP9 | − | 18-8/20-7:1:0 | GTTTGGCCCCTTCAGTGGAT |
| | | | | | CATGTTACCTATCA (SEQ ID |
| | | | | | NO: 21) |
| inhA | −15C→T | inh15_SSP10 | + | 18-14/14-6:1:0 | CTCGTGGACATACCGATTG |
| | | | | | TTGTTCATCAGTAGCGAGAT |
| | | | | | (SEQ ID NO: 22) |
| inhA | −15C→T | inh15_SSP11 | + | 18-14/13-7:1:0 | CTCGTGGACATACCGATTG |
| | | | | | TTGTTCATCAGTGGCGAGAT |
| | | | | | (SEQ ID NO: 23) |
| inhA | −15C→T | inh15_SSP12 | + | 18-14/8-6:1:0 | CTCGTGGACATACCGATTG |
| | | | | | TTGTTCAGCGAGAT (SEQ ID |
| | | | | | NO: 24) |
| inhA | −15C→T | inh15_SSP13 | + | 18-14/8-7:1:0 | CTCGTGGACATACCGATTG |
| | | | | | TTGTTCAGGCGAGAT (SEQ |
| | | | | | ID NO: 25) |
| inhA | −15C→T | inh15_SSP14 | − | 18-14/14-6:1:0 | CCCTTCAGTGGCTGTGGCTA |
| | | | | | TATTGTCATGTTCCTCTCA |
| | | | | | (SEQ ID NO: 26) |
| inhA | −15C→T | inh15_SSP15 | − | 18-14/13-7:1:0 | CCCTTCAGTGGCTGTGGCTA |
| | | | | | TATTGTCATGTTACCTCTCA |
| | | | | | (SEQ ID NO: 27) |
| inhA | −15C→T | inh15_SSP16 | − | 18-20/8-7:1:0 | CAGTGGCTGTGGCAGTCAT |
| | | | | | CTAGTACACTCGTCATGTTAC |
| | | | | | CTCTCA (SEQ ID NO: 28) |
| inhA | −15C→T | inh15_SSP17 | + | 20-14/14-6:1:0 | CGCTCGTGGACATACCGAT |
| | | | | | TGTTGTTCATCAGTAGCGAG |
| | | | | | AT (SEQ ID NO: 29) |
| inhA | −15C→T | inh15_SSP18 | + | 20-14/13-7:1:0 | CGCTCGTGGACATACCGAT |
| | | | | | TGTTGTTCATCAGTGGCGAG |
| | | | | | AT (SEQ ID NO: 30) |
| inhA | −15C→T | inh15_SSP19 | − | 18-14/14-6:1:2 | CCCTTCAGTGGCTGTGGCTA |
| | | | | | TATTGTCATGTTCCTATCAT |
| | | | | | C (SEQ ID NO: 31) |
| inhA | −15C→T | inh15_SSP20 | − | 18-14/13-7:1:2 | CCCTTCAGTGGCTGTGGCTA |
| | | | | | TATTGTCATGTTACCTATCA |
| | | | | | TC (SEQ ID NO: 32) |
| inhA | −15C→T | inh15_SSP21 | − | 18-20/8-7:1:2 | CAGTGGCTGTGGCAGTCAT |
| | | | | | CTAGTACACTCGTCATGTTAC |
| | | | | | CTATCATC (SEQ ID NO: 33) |
| inhA | −15C→T | inh15_SSP22 | − | 18-14/14-3:1:2 | TTCAGTGGCTGTGGCAGTA |
| | | | | | TTGTTACATTATAATCATC |
| | | | | | (SEQ ID NO: 34) |
| inhA | −15C→T | inh15_SSP23 | − | 18-14/14-3:2:1 | TTCAGTGGCTGTGGCAGTA |
| | | | | | TTGTTACATTATAATCACC |
| | | | | | (SEQ ID NO: 35) |
| inhA | −15C→T | inh15_SSP24 | − | 18-14/14-6:2:1 | CCCTTCAGTGGCTGTGGCTA |
| | | | | | TATTGTCATGTTCCTATCAC |
| | | | | | C (SEQ ID NO: 36) |
| inhA | −15C→T | inh15_SSP25 | − | 18-14/14-6:2:0 | CCCTTCAGTGGCTGTGGCTA |
| | | | | | TATTGTCATGTTCCTATCAC |
| | | | | | (SEQ ID NO: 37) |
| inhA | −15C→T | inh15_SSP26 | − | 18-10/14-6:1:0 | CCCTTCAGTGGCTGTGGCTA |
| | | | | | TATTGTCATGTTCCTATCA |
| | | | | | (SEQ ID NO: 38) |
| inhA | −15C→T | inh15_SSP27 | − | 20-14/13-7:1:0 | GCCCCTTCAGTGGCTGTGG |
| | | | | | CTATATTGTCATGTTACCTAT |
| | | | | | CA (SEQ ID NO: 39) |
| inhA | −15C→T | inh15_SSP28 | − | 18-14/12-8:1:0 | CCCTTCAGTGGCTGTGGCTA |
| | | | | | TATTGTCATGTTAACCTATC |
| | | | | | A (SEQ ID NO: 40) |
| inhA | −17G→T | inh17_SSP1 | − | 18-14/15-6:1:0 | CCTTCAGTGGCTGTGGCAT |
| | | | | | ATATTGTCATGTTTATCGTA |
| | | | | | (SEQ ID NO: 41) |
| inhA | −17G→T | inh17_SSP2 | − | 18-14/14-6:1:0 | CTTCAGTGGCTGTGGCAGT |
| | | | | | ATATTGTCATGTTTATCGTA |
| | | | | | (SEQ ID NO: 42) |
| inhA | −17G→T | inh17_SSP3 | − | 18-14/13-7:1:0 | CTTCAGTGGCTGTGGCAGT |
| | | | | | ATATTGTCATGTTCTATCGT |
| | | | | | A (SEQ ID NO: 43) |
| inhA | −17G→T | inh17_SSP4 | − | 20-14/14-6:1:0 | CCCTTCAGTGGCTGTGGCA |
| | | | | | GTATATTGTCATGTTTATCG |
| | | | | | TA (SEQ ID NO: 44) |
| inhA | −17G→T | inh17_SSP5 | − | 20-14/13-7:1:0 | CCCTTCAGTGGCTGTGGCA |
| | | | | | GTATATTGTCATGTTCTATC |
| | | | | | GTA (SEQ ID NO: 45) |
| inhA | | inhA_rev1 | + | | GGAAATCGCAGCCACGTTA |
| | | | | | C (SEQ ID NO: 46) |
| inhA | | inhA_rev2 | + | | TTACGCTCGTGGACATACC |
| | | | | | G (SEQ ID NO: 47) |
| inhA | | inhA_rev6 | + | | CGTAACCCCAGTGCGAAAG |
| | | | | | T (SEQ ID NO: 48) |
| inhA | | inhA_for2 | − | | CTCGTGGACATACCGATT |
| | | | | | (SEQ ID NO: 49) |
| inhA | | inhA_for4 | − | | CACGTTACGCTCGTGGACA |
| | | | | | T (SEQ ID NO: 50) |
| rpoB | D516V | rpoB516_SSP1 | − | 20-14/14-6:1:0 | TCGGCGCTTGTGGGTCAAC |
| | (GAC→GTC) | | | | CTGCTTGTATACACATTCTG |
| | | | | | GA (SEQ ID NO: 51) |
| rpoB | D516V | rpoB516_SSP2 | − | 18-14/14-6:1:0 | GGCGCTTGTGGGTCAACCT |
| | (GAC→GTC) | | | | GCTTGTATACACATTCTGGA |
| | | | | | (SEQ ID NO: 52) |
| rpoB | D516V | rpoB516_SSP3 | − | 20-14/13-7:1:0 | TCGGCGCTTGTGGGTCAAC |
| | (GAC→GTC) | | | | CTGCTTGTATACACAGTTCT |
| | | | | | GGA (SEQ ID NO: 53) |
| rpoB | D516V | rpoB516_SSP4 | − | 18-14/13-7:1:0 | GGCGCTTGTGGGTCAACCT |
| | (GAC→GTC) | | | | GCTTGTATACACAGTTCTGG |
| | | | | | A (SEQ ID NO: 54) |
| rpoB | H526D | rpoB526D_SSP1 | − | 20-14/14-6:1:0 | GACAGACCGCCGGGCCCCA |
| | (CAC→GAC) | | | | GTATTACAGTAAATTGCTTG |
| | | | | | TC (SEQ ID NO: 55) |
| rpoB | H526D | rpoB526D_SSP2 | − | 16-14/22-6:1:0 | ACGTGACAGACCGCCGTAT |
| | (CAC→GAC) | | | | TACAGTAAATTGCTTGTC |
| | | | | | (SEQ ID NO: 56) |
| rpoB | H526D | rpoB526D_SSP3 | − | 16-14/21-7:1:0 | ACGTGACAGACCGCCGTAT |
| | (CAC→GAC) | | | | TACAGTAAATTCGCTTGTC |
| | | | | | (SEQ ID NO: 57) |
| rpoB | H526D | rpoB526D_SSP4 | − | 18-14/14-6:1:0 | CAGACCGCCGGGCCCCAGT |
| | (CAC→GAC) | | | | ATTACAGTAAATTGCTTGTC |
| | | | | | (SEQ ID NO: 58) |
| rpoB | H526D | rpoB526D_SSP5 | − | 18-14/22-6:1:0 | TCACGTGACAGACCGCCGT |
| | (CAC→GAC) | | | | ATTACAGTAAATTGCTTGTC |
| | | | | | (SEQ ID NO: 59) |
| rpoB | H526D | rpoB526D_SSP6 | − | 20-14/13-7:1:0 | GACAGACCGCCGGGCCCCA |
| | (CAC→GAC) | | | | GTATTACAGTAAATTCGCTT |
| | | | | | GTC (SEQ ID NO: 60) |
| rpoB | H526D | rpoB526D_SSP7 | − | 18-14/13-7:1:0 | CAGACCGCCGGGCCCCAGT |
| | (CAC→GAC) | | | | ATTACAGTAAATTCGCTTGT |
| | | | | | C (SEQ ID NO: 61) |
| rpoB | H526D | rpoB526D_SSP8 | − | 18-14/21-7:1:0 | TCACGTGACAGACCGCCGT |
| | (CAC→GAC) | | | | ATTACAGTAAATTCGCTTGT |
| | | | | | C (SEQ ID NO: 62) |
| rpoB | H526Y | rpoB526Y_SSP1 | − | 20-14/14-6:1:0 | GACAGACCGCCGGGCCCCA |
| | (CAC→TAC) | | | | GTATTACAGTAAATTGCTTG |
| | | | | | TA (SEQ ID NO: 63) |
| rpoB | H526Y | rpoB526Y_SSP2 | − | 16-14/22-6:1:0 | ACGTGACAGACCGCCGTAT |
| | (CAC→TAC) | | | | TACAGTAAATTGCTTGTA |
| | | | | | (SEQ ID NO: 64) |
| rpoB | H526Y | rpoB526Y_SSP3 | − | 16-14/21-7:1:0 | ACGTGACAGACCGCCGTAT |
| | (CAC→TAC) | | | | TACAGTAAATTCGCTTGTA |
| | | | | | (SEQ ID NO: 65) |
| rpoB | H526Y | rpoB526Y_SSP4 | − | 18-14/14-6:1:0 | CAGACCGCCGGGCCCCAGT |
| | (CAC→TAC) | | | | ATTACAGTAAATTGCTTGTA |
| | | | | | (SEQ ID NO: 66) |
| rpoB | H526Y | rpoB526Y_SSP5 | − | 18-14/22-6:1:0 | TCACGTGACAGACCGCCGT |
| | (CAC→TAC) | | | | ATTACAGTAAATTGCTTGTA |
| | | | | | (SEQ ID NO: 67) |
| rpoB | H526Y | rpoB526Y_SSP6 | − | 20-14/13-7:1:0 | GACAGACCGCCGGGCCCCA |
| | (CAC→TAC) | | | | GTATTACAGTAAATTCGCTT |
| | | | | | GTA (SEQ ID NO: 68) |
| rpoB | H526Y | rpoB526Y_SSP7 | − | 18-14/13-7:1:0 | CAGACCGCCGGGCCCCAGT |
| | (CAC→TAC) | | | | ATTACAGTAAATTCGCTTGT |
| | | | | | A (SEQ ID NO: 69) |
| rpoB | H526Y | rpoB526Y_SSP8 | − | 18-14/21-7:1:0 | TCACGTGACAGACCGCCGT |
| | (CAC→TAC) | | | | ATTACAGTAAATTCGCTTGT |
| | | | | | A (SEQ ID NO: 70) |
| rpoB | S531L | rpoB531_SSP1 | − | 20-14/14-6:1:0 | CCCGGCACGCTCACGTGAC |
| | (TCG→TTG) | | | | ATGTTATACTAAATTAGCGC |
| | | | | | CA (SEQ ID NO: 71) |
| rpoB | S531L | rpoB531_SSP2 | − | 20-14/12-6:1:0 | CGGCACGCTCACGTGACAG |
| | (TCG→TTG) | | | | ATGTTATACTAAATTAGCGC |
| | | | | | CA (SEQ ID NO: 72) |
| rpoB | S531L | rpoB531_SSP3 | − | 18-14/14-6:1:0 | CGGCACGCTCACGTGACAT |
| | (TCG→TTG) | | | | GTTATACTAAATTAGCGCCA |
| | | | | | (SEQ ID NO: 73) |
| rpoB | S531L | rpoB531_SSP4 | − | 20-14/13-7:1:0 | CCCGGCACGCTCACGTGAC |
| | (TCG→TTG) | | | | ATGTTATACTAAATTCAGCG |
| | | | | | CCA (SEQ ID NO: 74) |
| rpoB | S531L | rpoB531_SSP5 | − | 20-14/11-7:1:0 | CGGCACGCTCACGTGACAG |
| | (TCG→TTG) | | | | ATGTTATACTAAATTCAGCG |
| | | | | | CCA (SEQ ID NO: 75) |
| rpoB | S531L | rpoB531_SSP6 | − | 18-14/13-7:1:0 | CGGCACGCTCACGTGACAT |
| | (TCG→TTG) | | | | GTTATACTAAATTCAGCGCC |
| | | | | | A (SEQ ID NO: 76) |
| rpoB | S531L | rpoB_rev1 | + | | GAGGCGATCACACCGCAGA |
| | (TCG→TTG) | | | | C (SEQ ID NO: 77) |
| rpoB | S531L | rpoB_rev2 | + | | GCCGCGATCAAGGAGTTCT |
| | (TCG→TTG) | | | | TC (SEQ ID NO: 78) |

2.6 Validation of RT-PCR Assays for Mutant Detection with Molecular Beacon Probes

Amplification of WT DNA can lead to a false positive sample detection in RT-PCR assays, particularly for clinical samples with a low number of copies of mutant DNA and a high number of copies of WT DNA. To confirm whether the amplification products (from WT DNA) in SuperSelective PCR assays were the intended amplification products and not the amplification of non-specific products, such as primer-dimers, the inventors designed and tested amplification-product-specific molecular beacon probes in the PCR assays [32]. Since non-hybridized molecular beacons are virtually non-fluorescent and hybridized molecular beacons fluoresce brightly in their characteristic color [32,33], the amount of amplicon present during the annealing phase of each amplification cycle is automatically measured by the spectrofluorometric thermal cycler in which the PCR assays are carried out.

### 2.7 Targeting Closely Localized Mutations

Of the panel of mutations selected for this study, both katG mutations AGC→ACA and AGC→ACC are present within the same codon (315). The anchor and foot sequences of the SuperSelective primers that were designed to detect these mutations overlapped with each other. Similarly, inhA mutations −8T→A, −15C→T, and −17G→T are co-localized in the promoter region, and rpoB mutations D516V (GAC→GTC), H526D (CAC→GAC), H526Y (CAC→TAC), and S531L (TCG→TTG) are co-localized in the RIF-resistance-determining region (RRDR) with overlapping regions of the primer. The amplification products of both katG mutations, three inhA promoter mutations, and four rpoB mutations also had overlapping nucleotide sequences. These regions were exploited to design the molecular beacon probes to detect the mutations. Thus, one could detect both katG mutations using the same/single probe. Similarly, the inventors designed one probe for the identification of three mutations in the inhA promoter region, i.e., −8T→A, −15C→T, −17G→T, and one probe for all of the rpoB mutations D516V (GAC→GTC), H526D (CAC→GAC), H526Y (CAC→TAC), and S531L (TCG→TTG). Thus, three molecular beacon probes were utilized to detect nine mutations. The inventors designed the following probes, which included a unique sequence at both ends of the probe (shown in italics), to form the stem region and labeled the probe with FAM at the 5′ end and a quencher BHQ-1 at the 3′ end. The respective loop regions (SEQ ID NOs: 2, 4, and 6) are shown in bold.

katG:
(SEQ ID NO: 1)
5′ FAM-CGCTCG GACGAACACCCCGACGAAAT CGAGCG-BHQ-1 3′
inhA:
(SEQ ID NO: 3)
5′ FAM-CGCTCG CCGGGCCGAAATCGGTATGT CGAGCG-BHQ-1 3′
rpoB:
(SEQ ID NO: 5)
5′ FAM-CGCTCG GAATTGGCTCAGCTGGCTGG CGAGCG-BHQ-1 3′

### 2.8. Use of Asymmetric POR Conditions

Three separate RT-PCR assays were conducted to optimize the molecular beacon probe concentrations for katG, inhA, and rpoB. The concentration of each probe was optimized for at least one mutation from the group for which it was specific. Thus, molecular beacon probe katG was optimized for the detection of the katG mutation S315T (AGC→ACA), the inhA probe was optimized for the detection of the inhA promoter mutation-8T→A, and the rpoB probe was optimized for the detection of the rpoB mutation D516V (GAC→GTC). RT-PCR assays were carried out using 10⁵ copies of linearized mutant plasmid in three sets of reactions with variable concentrations of the probe, i.e., 0.1, 0.125, and 0.25 μM. 0.1 μM each of the forward and reverse primers were used in these assays. The fluorescence intensity with the 0.25 μM probe was the highest as expected (>2-fold), while there was no significant difference between the relative fluorescence units (RFU) obtained with 0.1 and 0.125 μM probes. FIG. 6 demonstrates an example of the increase in fluorescence signal intensity with increasing concentration of the inhA probe with 10⁵ copies of inhA −8T→A mutants, using SuperSelective primer 18-14/14-6:1:0.

In order to avoid probe crosstalk in multiplex reactions with a very high amount of probe, the inventors tested asymmetric primer concentrations to enhance fluorescence signal intensity in the reactions to control the amount of molecular beacon probes. RT-PCR assays were performed in two sets with a SuperSelective primer and a conventional reverse primer in the ratios of 1:5 and 1:10 on 10⁵ copies of mutant target DNA using a 0.1 μM probe. SuperSelective primer and conventional reverse primer in the ratio 1:1 (symmetric PCR, 0.1 μM each of forward and reverse primer) were used as a control for these reactions. The inventors observed a 2.5- to 3-fold rise in fluorescence signal intensity under both tested asymmetric PCR conditions, although there was no significant difference between primer ratios 1:5 and 1:10. FIG. 7 shows the amplification of rpoB D516V GAC→GTC with SuperSelective primer 18-14/13-7:1:0 paired with a conventional reverse primer in 1:1, 1:5, and 1:10 ratios on 10⁵ copies of mutant target DNA. These experiments demonstrate the advantage of using non-symmetric primer concentrations in RT-PCR assays compared to the use of symmetric primer concentrations.

The inventors extended the observations from the above experiments to other mutations included in this study, katG S315T AGC→ACC, inhA promoter −15C→T and −17G→T, rpoB H526D (CAC→GAC), H526Y (CAC→TAC), and S531L (TCG→TTG). All of the subsequent monoplex RT-PCR assays with molecular beacon probes were performed under asymmetric PCR conditions utilizing 0.1 μM and 0.5 μM of forward and reverse primer, respectively, and 0.1 μM of the probe to maximize the fluorescence signal intensity. The inventors further optimized the individual probe concentrations for use in the multiplexed assays (discussed below).

2.9. Selective Amplification of the Mutant from Mixed DNA

To detect each of the nine mutations in the selected panel of target mutant genes, and to determine their selectivity, one to five SuperSelective primers were tested. RT-PCR experiments were performed using linearized mutant and WT pDNA templates in the ratios 10⁵ to 1:10⁵ (mutant: WT) and 10⁵ to 1:10⁴ (mutant: WT). Each primer was tested at least twice on duplicate samples. Low copy number mutants (ten and one) and WT samples were tested in three to eight replicates.

RT-PCR assays with the molecular beacon probes confirmed that the amplification products initiated from WT DNA templates (with 10⁵ copies of WT DNA) resulted from specific amplification. Under the tested assay conditions, SuperSelective primers successfully suppressed the amplification of 10⁴ copies of WT DNA while amplifying the mutant to one copy (Table 3). Primers were tested for up to 60 RT-PCR cycles to check for the appearance of late amplification signals (after Ct 50) from the wild type. Using the selected SuperSelective primers (Table 3), the inventors could detect all nine mutations in the panel with absolute specificity. The inventors could detect down to one copy of mutant within 50 cycles of the RT-PCR, with the detection of WT (up to 10⁴ copies) suppressed until 60 cycles. These observations confirmed that SuperSelective primers are highly specific for the amplification of the target mutant DNA (specificity 100%) while suppressing the amplification of up to 10⁴ copies of the closely related wild-type DNA.

TABLE 3
Limit of detection (LoD) of mutant DNA from clinical isolates.

| | | | | No. of |
|---|---|---|---|---|
| | SuperSelective | Conventional | | gDNA |
| | Forward | Reverse | | Samples |
| Mutation | Primer | Primer | LoD * | Tested |
| katG (AGC→ACC) | 20-14/14-5:1:0 | katG_rev2 | 1 | 7 |
| katG (AGC→ACA) | 20-14/14-5:2:0 | katG_rev2 | 1 | 2 |
| inhA −8T→A | 20-14/14-6:1:0 | inhA_rev1 | 1 | 2 |
| inhA −15C→T | 18-14/13-7:1:0 | inhA_rev1 | 1 | 4 |
| inhA −17G→T | 20-14/13-7:1:0 | inhA_rev2 | 1 | 1 |
| D516V (GAC→GTC) | 20-14/13-7:1:0 | rpoB_rev1 | 1 | 3 |
| H526D (CAC→GAC) | 20-14/13-7:1:0 | rpoB_rev1 | 1 | 2 |
| H526Y (CAC→TAC) | 20-14/13-7:1:0 | rpoB_rev1 | 1 | 2 |
| S531L (TCG→TTG) | 20-14/13-7:1:0 | rpoB_rev1 | 1 | 4 |

* LoD: The lowest number of detectable copies of mutant DNA in a background of 10⁴ copies of WT DNA.

2.10. Highly Sensitive Method of Mutant Detection in a Background of Susceptible M. tuberculosis DNA

The parameters of SuperSelective primer design and RT-PCR assay conditions were initially optimized using linearized plasmids containing the mutation of interest. Plasmids, which are limited in their total length, generate a low probability that SuperSelective primers will find non-specific binding sites, though this probability will increase many-fold in a sample containing copies of the entire genome of M. tuberculosis. To validate SuperSelective primer designs and RT-PCR assay conditions, the inventors tested selected SuperSelective forward primer and conventional reverse primer combinations (Table 3) on several M. tuberculosis genomic DNA samples containing the mutations of interest. The inventors tested these conditions on 15 mutant genomic DNA samples containing the panel of mutations, as shown in Table 3. Of these, ten genomic DNA samples were multidrug-resistant with the katG S315T/inhA-15/inhA-17 mutation and one of the four Rifampicin Resistance Determining Region (RRDR) mutations (Table 3).

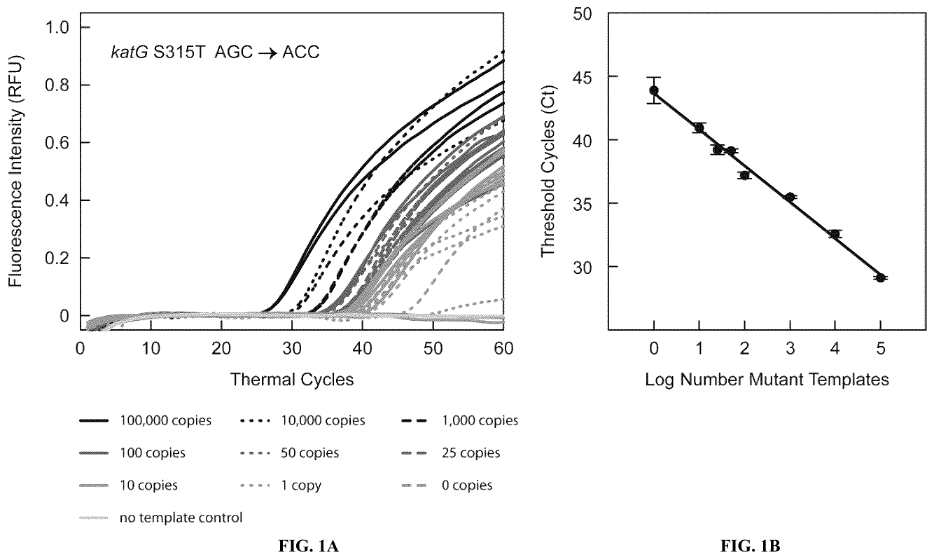

The inventors tested each of the selected SuperSelective primer and RT-PCR assay conditions on 10⁵ to one copy of mutant gDNA in a background of 10⁴ copies of wild-type M. tuberculosis H37Rv genomic DNA. The inventors tested two replicates for high copy numbers (10⁵, 10⁴, 10³, 10², 50) of mutant, eight replicates for the low copy numbers (25, 10, 1) of mutant, and at least four replicates of 10⁴ copies of wild type. Consistent with the findings in the plasmid-based assays (Table 3), the inventors were able to amplify up to one copy of mutant DNA (in at least two out of eight replicates) for all of the nine mutations in katG, inhA, and rpoB. Ten copies of mutant DNA were amplified in at least four out of eight replicates and twenty-five copies in at least six out of eight replicates, while eight out of eight replicates were positively detected for fifty copies of mutant DNA or more. FIG. 1A shows the amplification peaks obtained in RT-PCR assays containing 10⁵, 10⁴, 10³, 10², fifty, twenty-five, ten, one, and zero copies of a katG S315T AGC→ACC mutant gDNA sample in a background of 10⁴ copies of wild-type M. tuberculosis H37Rv DNA, utilizing SuperSelective primer 20-14/14-5:1:0 combined with a conventional reverse primer, and using a FAM-labeled molecular beacon probe for detection.

Thus, the above-described SuperSelective primer-based RT-PCR assays could detect one in ten thousand copies of mutant DNA for all nine gene mutations included in the panel, confirming the ultra-high sensitivity (0.01%) of these SuperSelective primers for the detection of INH and RIF resistance.

### 2.11. Quantifiable Mutant Detection

The clinical goal of these RT-PCR assays that utilize SuperSelective primers is to measure the relative abundance of mutant DNA fragments relevant to INH and RIF resistance in TB in the context of the amount of wild-type M. tuberculosis DNA present in the sample. In the RT-PCR assays, control reactions that contained no template DNA did not produce any false amplicons, such as primer-dimers, despite the longer length of the SuperSelective primers. The reaction containing 10⁴ wild-type templates and no mutant templates was suppressed to such an extent that it did not produce a significant number of amplicons until after 50 cycles of amplification had been carried out (FIG. 1A).

To examine the potential of SuperSelective primer pairs for mutant quantification, the inventors compared the mean Ct values (in an RT-PCR assay) with each SuperSelective primer to the mutant copy number tested. The mean Ct values were inversely linearly proportional to the logarithm of the number of copies of mutant target DNA (FIG. 1B). This inverse linear relationship between the logarithm of the number of mutant targets initially present in a sample and the Ct value observed for that sample is the hallmark of quantitative exponential amplification assays (see for example, Lomeli et al. (1989) Clin. Chem. 358:1826-1831). Thus, RT-PCR assays employing SuperSelective primer pairs could be utilized to determine the actual amount of mutant target DNA, even in a high background (10⁴ copies) of wild-type DNA, based only on Ct values. These results demonstrate the potential of SuperSelective primer in RT-PCR assays to not only detect, but also to quantify the amount of rare mutant DNA in the presence of abundant wild-type M. tuberculosis DNA, providing a novel and powerful tool for detecting and quantifying heteroresistance in TB patients.

### 2.12. Multiplexed Assay

The mutations causing INH and RIF resistance in M. tuberculosis are localized within a compact region of the katG gene, inhA promoter, and the RRDR of the rpoB gene. For mutant sequences occurring in separate genes, multiplex real-time PCR assays can be designed relatively easily to quantitate the amount of each mutant gene in the same sample. SuperSelective primers for these mutations will have different anchor and foot sequences. The amplicons generated from each mutant gene will possess a unique sequence that can be easily distinguished from the sequences of amplicons generated from the other mutant genes, making it easy to distinguish these amplicons from each other through the use of differently colored molecular beacon probes. Thus, the inventors decided to multiplex the RT-PCR assays to detect mutations present in different drug-resistance genes.

Amongst the drug-resistant TB cases, the katG S315T mutation is the most common mutation and the inhA mutation (−15C→T) is the second most frequently observed mutation associated with INH resistance [34], while the rpoB mutation S531L (TCG→TTG) is the most widespread mutation responsible for high-level resistance to RIF [35]. Considering the importance of these three mutations in MDR-TB cases, a prototype multiplex assay incorporating SuperSelective primers was designed to simultaneously detect the katG gene S315T AGC→ACA, the inhA-15 C→T, and the rpoB S531L TCG→TTG gene.

### 2.13. Optimization of Assay Components

For the simultaneous detection of the katG gene S315T AGC→ACA, the inhA −15 C→T gene, and the rpoB S531L TCG→TTG gene, the inventors utilized SuperSelective primers having an LoD of one copy of mutant in the corresponding monoplex assays (Table 3) and they tested the efficiency and specificity of these primers in a multiplexed environment. To ensure that the selected primer pairs do not have any cross-reactivity with each other, the inventors tested these three primer pairs in various possible combinations. For example, the inventors tested the katG S315T AGC→ACA and the inhA-15 C→T primer pair in a duplex RT-PCR assay utilizing 10⁵ copies of the katG S315T AGC→ACA mutation containing linearized pDNA to study the effect of the inhA-15 C→T primer pair addition on the amplification threshold of the katG S315T AGC→ACA mutant. A monoplex RT-PCR assay with a katG S315T AGC→ACA primer pair and 10⁵ copies of the katG S315T mutation contained in linearized pDNA was used as a control. SYBR Green was used to monitor the amplification of these reactions. The inventors also studied the effect of the katG S315T AGC→ACA primer pair addition on the amplification threshold of the inhA −15 C→T gene by using the inhA-15 C→T mutation contained in linearized pDNA.

Similarly, katG-rpoB, inhA-rpoB, and katG-inhA-rpoB primer pairs were also tested. The rpoB primer pair addition delayed the amplification threshold of both the katG S315T AGC→ACA and the inhA-15 C→T mutants in the duplex assays, but the Ct values in the three-plex assays were unaffected. Thus, these three primer pairs were utilized to further optimize the multiplex RT-PCR assay. For these three-plex RT-PCR assays, the inventors used three molecular beacon probes, each specific for amplicons generated by SuperSelective primers corresponding to katG, inhA, and rpoB. Each probe was labeled with a different colored fluorophore to detect signals for each mutation in a different optical channel.

katG:
(SEQ ID NO: 1)
5′ FAM-CGCTCG GACGAACACCCCGACGAAAT CGAGCG-BHQ-1 3′
inhA:
(SEQ ID NO: 3)
5′ Cal Fluor Red 610-CGCTCG CCGGGCCGAAATCGGTATGT
CGAGCG-BHQ-2 3′
rpoB:
(SEQ ID NO: 5)
5′ Quasar 670-CGCTCG GAATTGGCTCAGCTGGCTGG CGAGCG-
BHQ-2 3′

Probe concentrations were re-optimized for the multiplex RT-PCR assays. Asymmetric RT-PCR assays were carried out on a Bio-Rad CFX96 Real-Time System in a final volume of 25 μL with 1× PCR buffer supplemented with 25 mM TMAC and 0.25% Tween20, 3 mM MgCl₂, 0.05 U/μL Platinum Taq DNA polymerase, 0.25 mM dNTPs, each forward primer (katG, inhA, rpoB) 0.1 μM, each reverse primer (katG, inhA, rpoB) 0.5 μM, probe katG 0.25 μM, probe inhA 0.1 μM, and probe rpoB 0.25 μM. Amplification was performed for 60 cycles with Tm 60° C. for 20 s and extension at 72 for 20 s.

### 2.14. Selective Amplification

To ensure that each amplicon is only copied by its “correct” SuperSelective primer under the three-plex assay conditions, the inventors carried out three sets of experiments, each with linearized plasmids containing the katG S315T AGC→ACA, inhA-15 C→T, or rpoB TCG→TTG mutation. The inventors tested 10⁵-1:10⁴ copies of mutant: WT pDNA. Each condition was tested on at least two replicates.

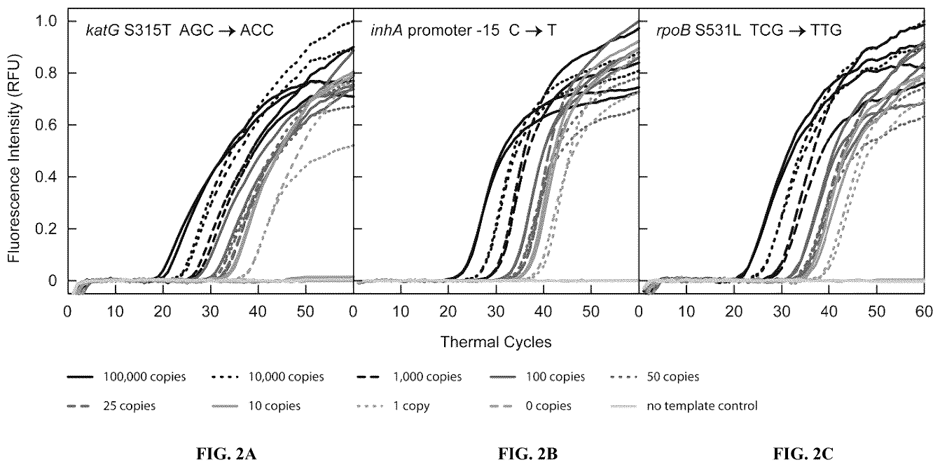

In the first set of RT-PCR multiplex assays, with the katG S315T AGC→ACA mutant-containing pDNA, the inventors detected amplification signals only in the FAM channel (FIG. 2A) down to one copy of mutant DNA in a background of 10⁴ copies of WT DNA. No signals were detected in the CAL Fluor Red 610 (CFR610) and the Quasar 670 (Q670) channels. Similarly, in multiplex reactions with pDNA containing the inhA-15 C→T and the rpoB S531L TCG→TTG mutant, fluorescence was recorded only in the CFR610 (FIG. 2B) and Q670 (FIG. 2C) channels, respectively, while no amplification signal was observed in the non-specific channels. These results confirmed that the SuperSelective primers detect only specific mutants even in the presence of abundant WT DNA fragments. None of the primer pairs cross-reacted to amplify a non-specific target, and they completely suppressed the unintended WT target in the background, signifying a high specificity of SuperSelective primers even in a multiplexed environment.

2.15. Sensitivity on Clinical gDNA with Potential Sputum Carryover

To expand the utility of optimized SuperSelective PCR to encompass clinical applications, the inventors tested 23 M. tuberculosis gDNA samples isolated from the sputum of pulmonary TB patients. The sputum samples were obtained from microbiologically, radiologically, and clinically confirmed TB cases (Table 4). These M. tuberculosis gDNA samples were single blinded as to the status of their phenotypic or genotypic drug resistance.

TABLE 4
Detection of mutations in clinical samples using SuperSelective primers.

| | | | katG (AGC > ACC) | | inhA (−15C→T) | | rpaB (S531L) | |
|---|---|---|---|---|---|---|---|---|
| | | | Number of | | Number of | | Number of | |
| | | | Mutant | | Mutant | | Mutant | |
| | | Total | DNA Copies | | DNA Copies | | DNA Copies | |
| | | DNA | Detected by | DNA | Detected by | DNA | Detected by | DNA |
| | | (rrs | SuperSelec- | Sequencing | SuperSelec- | Sequencing | SuperSelec- | Sequencing |
| Sample | Age/Sex | Copies) | tive Primers | Result | tive Primers | Result | tive Primers | Result |
| AVR1 | 36/M | 2595 | 2328 | AGC > ACC | 40 | WT | 2167 | S531L |
| AVR2 | 28/M | 254 | 5 | WT | 38 | WT | 284 | S531L |
| AVR4 | 39/F | 498 | 679 | — | 0 | — | 0 | — |
| AVR7 | 33/M | 540 | 1086 | AGC > ACC | 0 | WT | 1332 | S531L |
| AVR8 | 49/M | 979 | 601 | — | 0 | — | 0 | — |
| AVR9 | 37/F | 2867 | 2039 | AGC > ACC | 0 | WT | 2827 | S531L |
| AVR11 | 62/F | 24,693 | 2 | WT | 0 | WT | 4 | WT |
| AVR12 | 28/F | 3294 | 2262 | AGC > ACC | 0 | WT | 3992 | S531L |
| AVR13 | 24/F | 32 | 33 | AGC > ACC | 0 | WT | 42 | S531L |
| AVR14 | 70/F | 129 | 36 | AGC > ACC | 0 | WT | 57 | S531L |
| AVR16 | 26/M | 2038 | 534 | AGC > ACC | 0 | WT | 1184 | S531L |
| AVR18 | 27/F | 2335 | 0 | — | 84 | — | 0 | — |
| AVR19 | 42/M | 2680 | 0 | — | 0 | — | 0 | — |
| AVR23 | 26/F | 2867 | 0 | — | 0 | — | 0 | — |
| AVR24 | 19/F | 2434 | 0 | — | 0 | — | 0 | — |
| AVR25 | 38/F | 238 | 350 | — | 0 | — | 0 | — |
| AVR26 | 18/F | 197 | 197 | AGC > ACC | 0 | WT | 208 | S531L |
| AVR27 | 26/F | 2857 | 0 | — | 0 | — | 0 | — |
| AVR30 | 28/M | 1617 | 3885 | — | 0 | — | 0 | — |
| AVS32 | 18/M | 855 | 0 | — | 0 | — | 0 | — |
| AVS34 | 42/F | 3758 | 0 | — | 0 | — | 0 | — |
| AVS35 | 57/M | 5559 | 0 | WT | 10 | WT | 2 | WT |
| AVS38 | 35/F | 280 | 38 | — | 0 | — | 0 | — |

The Inventors first assessed the total amount of DNA present in each of these 23 clinical gDNA samples by amplifying a reference M. tuberculosis wild-type gene sequence, 16S rRNA (rrs), present in the sample and unrelated to the mutations of interest. The generation of amplicons from this reference gene sequence served as a quantitative indicator control for the gDNA samples. The RT-PCR assays were performed using the conventional forward and reverse primer to amplify a region of the rrs gene using SYBR Green to monitor amplification. The Ct value of these WT amplicons reflects the amount of DNA present in the sample. The number of copies of rrs was calculated using the slope and intercept of the standard curve, which was plotted using 10⁵-1 copies of WT H37Rv gDNA. If the number of genomes turned out to be zero or lower than a predetermined value, the assay results would have been ignored due to there being too little DNA in the sample for the rare target mutations, if they exist, to be present.

This assay confirmed that all the clinical samples had detectable copies of the rrs, ranging from 33 to 25,000. Next, the inventors performed RT-PCR using the mutation-specific SuperSelective primers (Table 3) to determine the number of mutant DNA copies present in each of these 23 clinical M. tuberculosis gDNA samples, with mutant gDNA used to prepare the standards at copy numbers ranging from 1 to 10⁵. The inventors first plotted the standard curve using mean Ct values versus the logarithm of the number of copies of mutant target DNA for each mutation (as shown in FIG. 1B). Next, the clinical M. tuberculosis gDNA samples were subjected to RT-PCR with the SuperSelective primers, and the number of copies of each mutant gene was calculated based on the slope and intercept of the standard curve.

To validate the assessment of drug-resistance detection based on SuperSelective primers, sections of the katG, inhA, and rpoB genes containing the mutations of interest were sequenced in 11 of the 23 samples (Table 4). Eight of the twenty-three samples were also tested for phenotypic drug susceptibility using phenotypic drug-susceptibility testing (pDST). Based on the RT-PCR assay using SuperSelective primers, six of the twenty-three samples were found to be wild type, two of which were also confirmed by pDST. Using SuperSelective primers, six of the twenty-three samples were found to be INH-monoresistant, including five with the katG S315T AGC→ACA mutation and one with the inhA −15 C→T mutation. Of these, one of the katG mutant samples was confirmed to be INH-resistant by pDST. Using SuperSelective primers, the inventors also identified 11 of the 23 clinical genomic DNA samples as multidrug-resistant with mutations conferring both INH and RIF resistance. Five of eleven of these samples had a complete agreement between the SuperSelective primer RT-PCR assay and sequencing, and three of the eleven samples tested by pDST were confirmed to be INH-resistant and RIF-resistant (Table 4). In three of the eleven samples (AVR2, AVR11, and AVS35), there was a discordance between the SuperSelective primer RT-PCR assay and sequencing. The SuperSelective primer RT-PCR assay detected sample AVR2 as multidrug-resistant, demonstrating five copies of katG S315T AGC→ACA and thirty-eight copies of inhA −15 C→T mutant DNA in a total sample containing two hundred fifty-four gDNA copies. Neither of these INH mutations was detected by DNA sequencing; thus, this sample would be considered to be RIF-monoresistant, rather than multidrug resistant. With the SuperSelective primer RT-PCR assay, sample AVR11 had two copies of the katG S315T AGC→ACA mutant DNA and four copies of the rpoB S531L TCG→TTG mutant DNA, in a total background of over twenty-four thousand copies of gDNA. This sample was identified as wild type by sequencing, yet the pDST results of this sample classified it as multidrug resistant. In another sample (AVS35), pDST and sequencing failed to detect drug resistance. However, SuperSelective primers identified the presence of ten copies of inhA −15 C→T mutant DNA and two copies of rpoB S531L TCG→TTG mutant DNA, with rrs copies being >5,500 in this sample.

While targeted sequencing is known to detect heteroresistance with a limit of detection (LoD) of 10%, SuperSelective primers were able to detect specific mutants as low as two copies in the presence of abundant DNA fragments from the entire M. tuberculosis genome with exceptional sensitivity, due to the suppression of amplification of the abundant wild-type DNA fragments.

### REFERENCES

1. World Health Organization Global Tuberculosis Report; WHO: Geneva, Switzerland, 2021; Volume 57.
2. Gygli, S. M.; Borrell, S.; Trauner, A.; Gagneux, S. Antimicrobial resistance in Mycobacterium tuberculosis: Mechanistic and evolutionary perspectives. FEMS Microbiol. Rev. 2017, 41, 354-373.
3. Law, S.; Piatek, A. S.; Vincent, C.; Oxlade, O.; Menzies, D. Emergence of drug resistance in patients with tuberculosis cared for by the Indian health-care system: A dynamic modelling study. Lancet Public Health 2017, 2, e47-e55.
4. Seaworth, B. J.; Griffith, D. E. Therapy of multidrug-resistant and extensively drug-resistant tuberculosis. Microbiol. Spectr. 2017, vol. 5, No. 2.
5. Schwoebel, V.; Trebucq, A.; Kashongwe, Z.; Bakayoko, A. S.; Kuaban, C.; Noeske, J.; Harouna, S. H.; Souleymane, M. B.; Piubello, A.; Ciza, F.; et al. Outcomes of a nine-month regimen for rifampicin-resistant tuberculosis up to 24 months after treatment completion in nine African countries. eClinicalMedicine 2020, 20, 100268.
6. Dheda, K.; Barry, C. E., 3rd; Maartens, G. Tuberculosis. Lancet 2016, 387, 1211-1226.
7. Palaci, M.; Dietze, R.; Hadad, D. J.; Ribeiro, F. K.; Peres, R. L.; Vinhas, S. A.; Maciel, E. L.; do Valle Dettoni, V.; Horter, L.; Boom, W. H.; et al. Cavitary disease and quantitative sputum bacillary load in cases of pulmonary tuberculosis. J. Clin. Microbiol. 2007, 45, 4064-4066.
8. Chen, L.; Zhang, J.; Zhang, H. Heteroresistance of Mycobacterium tuberculosis strains may be associated more strongly with poor treatment outcomes than within-host heterogeneity of M. tuberculosis infection. J. Infect. Dis. 2016, 214, 1286-1287.
9. Eilertson, B.; Maruri, F.; Blackman, A.; Herrera, M.; Samuels, D. C.; Sterling, T. R. High proportion of heteroresistance in gyrA and gyrB in fluoroquinolone-resistant Mycobacterium tuberculosis clinical isolates. Antimicrob. Agents Chemother. 2014, 58, 3270-3275.
10. Pholwat, S.; Stroup, S.; Foongladda, S.; Houpt, E. Digital PCR to detect and quantify heteroresistance in drug resistant Mycobacterium tuberculosis. PLOS ONE 2013, 8, e57238.
11. Rinder, H.; Mieskes, K. T.; Loscher, T. Heteroresistance in Mycobacterium tuberculosis. Int. J. Tuberc. Lung Dis. 2001, 5, 339-345.
12. Ng, K. C. S.; Supply, P.; Cobelens, F. G. J.; Gaudin, C.; Gonzalez-Martin, J.; de Jong, B. C.; Rigouts, L. How well do routine molecular diagnostics detect rifampin heteroresistance in Mycobacterium tuberculosis? J. Clin. Microbiol. 2019, 57, e00717-19.
13. Tolani, M. P.; D'Souza, D. T.; Mistry, N. F. Drug resistance mutations and heteroresistance detected using the Genotype MTBDRplusassay and their implication for treatment outcomes in patients from Mumbai, India. BMC Infect. Dis. 2012, 12, 9.
14. Folkvardsen, D. B.; Thomsen, V. O.; Rigouts, L.; Rasmussen, E. M.; Bang, D.; Bernaerts, G.; Werngren, J.; Toro, J. C.; Hoffner, S.; Hillemann, D.; et al. Rifampin heteroresistance in Mycobacterium tuberculosis cultures as detected by phenotypic and genotypic drug susceptibility test methods. J. Clin. Microbiol. 2013, 51, 4220-4222.
15. Maningi, N. E.; Daum, L. T.; Rodriguez, J. D.; Mphahlele, M.; Peters, R. P.; Fischer, G. W.; Chambers, J. P.; Fourie, P. B. Improved detection by next-generation sequencing of pyrazinamide resistance in Mycobacterium tuberculosis isolates. J. Clin. Microbiol. 2015, 53, 3779-3783.
16. Zhang, Z.; Wang, Y.; Pang, Y.; Liu, C. Comparison of different drug susceptibility test methods to detect rifampin heteroresistance in Mycobacterium tuberculosis. Antimicrob. Agents Chemother. 2014, 58, 5632-5635.
17. Ye, M.; Yuan, W.; Molaeipour, L.; Azizian, K.; Ahmadi, A.; Kouhsari, E. Antibiotic heteroresistance in Mycobacterium tuberculosis isolates: A systematic review and meta-analysis. Ann. Clin. Microbiol. Antimicrob. 2021, 20, 73.
18. Cohen, T.; Chindelevitch, L.; Misra, R.; Kempner, M. E.; Galea, J.; Moodley, P.; Wilson, D. Within-host heterogeneity of Mycobacterium tuberculosis infection is associated with poor early treatment response: A prospective cohort study. J. Infect. Dis. 2016, 213, 1796-1799.
19. Shin, S. S.; Modongo, C.; Zetola, N. M. The impact of mixed infections on the interpretation of molecular epidemiology studies of tuberculosis. Int. J. Tuberc. Lung Dis. 2016, 20, 423-424.
20. Zetola, N. M.; Shin, S. S.; Tumedi, K. A.; Moeti, K.; Ncube, R.; Nicol, M.; Collman, R. G.; Klausner, J. D.; Modongo, C. Mixed Mycobacterium tuberculosis complex infections and false-negative results for rifampin resistance by GeneXpert MTB/RIF are associated with poor clinical outcomes. J. Clin. Microbiol. 2014, 52, 2422-2429.
21. Mendez, M. P.; Landon, M. E.; McCloud, M. K.; Davidson, P.; Christensen, P. J. Co-infection with pansensitive and multidrug-resistant strains of Mycobacterium tuberculosis. Emerg. Infect. Dis. 2009, 15, 578-580.
22. Hingley-Wilson, S. M.; Casey, R.; Connell, D.; Bremang, S.; Evans, J. T.; Hawkey, P. M.; Smith, G. E.; Jepson, A.; Philip, S.; Kon, O. M.; et al. Undetected multidrug-resistant tuberculosis amplified by first-line therapy in mixed infection. Emerg. Infect. Dis. 2013, 19, 1138-1141.
23. Xu, Y.; Wu, J.; Liao, S.; Sun, Z. Treating tuberculosis with high doses of anti-TB drugs: Mechanisms and outcomes. Ann. Clin. Microbiol. Antimicrob. 2017, 16, 67.
24. Devaleenal Daniel, B.; Ramachandran, G.; Swaminathan, S. The challenges of pharmacokinetic variability of first-line anti-TB drugs. Expert Rev. Clin. Pharmacol. 2017, 10, 47-58.
25. Vargas, D. Y.; Kramer, F. R.; Tyagi, S.; Marras, S. A. Multiplex real-time PCR assays that measure the abundance of extremely rare mutations associated with cancer. PLOS ONE 2016, 11, e0156546.
26. Newton, C. R.; Graham, A.; Heptinstall, L. E.; Powell, S. J.; Summers, C.; Kalsheker, N.; Smith, J. C.; Markham, A. F. Analysis of any point mutation in DNA. The amplification refractory mutation system (ARMS). Nucleic Acids Res. 1989, 17, 2503-2516.
27. Chun, J. Y.; Kim, K. J.; Hwang, I. T.; Kim, Y. J.; Lee, D. H.; Lee, I. K.; Kim, J. K. Dual priming oligonucleotide system for the multiplex and SNP genotyping of CYP2C19 gene. Nucleic Acids Res. 2007, 35, e40.
28. Hazbon, M. H.; Alland, D. Hairpin primers for simplified single-nucleotide polymorphism analysis of Mycobacterium tuberculosis and other organisms. J. Clin. Microbiol. 2004, 42, 1236-1242.
29. Tabrizi, S. N.; Tan, L. Y.; Walker, S.; Twin, J.; Poljak, M.; Bradshaw, C. S.; Fairley, C. K.; Bissessor, M.; Mokany, E.; Todd, A. V.; et al. Multiplex assay for simultaneous detection of Mycoplasma genitalium and macrolide resistance using PlexZyme and PlexPrime technology. PLOS ONE 2016, 11, e0156740.
30. Cole, S. T.; Brosch, R.; Parkhill, J.; Garnier, T.; Churcher, C.; Harris, D.; Gordon, S. V.; Eiglmeier, K.; Gas, S.; Barry, C. E., 3rd;
   et al. Deciphering the biology of Mycobacterium tuberculosis from the complete genome sequence. Nature 1998, 393, 537-544.
31. Kramer, F. R.; Vargas, D. Y. SuperSelective primer pairs for sensitive detection of rare somatic mutations. Sci. Rep. 2021, 11, 22384.
32. Tyagi, S.; Kramer, F. R. Molecular beacons: Probes that fluoresce upon hybridization. Nat. Biotechnol. 1996, 14, 303-308.
33. Tyagi, S.; Bratu, D. P.; Kramer, F. R. Multicolor molecular beacons for allele discrimination. Nat. Biotechnol. 1998, 16, 49-53.
34. Seifert, M.; Catanzaro, D.; Catanzaro, A.; Rodwell, T. C. Genetic mutations associated with isoniazid resistance in Mycobacterium tuberculosis: A systematic review. PLOS ONE 2015, 10, e0119628.
35. Somoskovi, A.; Parsons, L. M.; Salfinger, M. The molecular basis of resistance to isoniazid, rifampin, and pyrazinamide in Mycobacterium tuberculosis. Respir. Res. 2001, 2, 164-168.
36. Hofmann-Thiel, S.; van Ingen, J.; Feldmann, K.; Turaev, L.; Uzakova, G. T.; Murmusaeva, G.; van Soolingen, D.; Hoffmann, H. Mechanisms of heteroresistance to isoniazid and rifampin of Mycobacterium tuberculosis in Tashkent, Uzbekistan. Eur. Respir. J. 2009, 33, 368-374.
37. Rosales-Klintz, S.; Jureen, P.; Zalutskayae, A.; Skrahina, A.; Xu, B.; Hu, Y.; Pineda-Garcia, L.; Merza, M. A.; Muntean, I.; Bwanga, F.; et al. Drug resistance-related mutations in multidrug-resistant Mycobacterium tuberculosis isolates from diverse geographical regions. Int. J. Mycobacteriol. 2012, 1, 124-130.
38. Vargas, D. Y.; Marras, S. A. E.; Tyagi, S.; Kramer, F. R. Suppression of wild-type amplification by selectivity enhancing agents in PCR assays that utilize SuperSelective primers for the detection of rare somatic mutations. J. Mol. Diagn. 2018, 20, 415-427.
39. Zuker, M. Mfold web server for nucleic acid folding and hybridization prediction. Nucleic Acids Res. 2003, 31, 3406-3415.
40. Weeks, S. D.; Drinker, M.; Loll, P. J. Ligation independent cloning vectors for expression of SUMO fusions. Protein Expr. Purif. 2007, 53, 40-50.
41. Pym, A. S.; Saint-Joanis, B.; Cole, S. T. Effect of katG mutations on the virulence of Mycobacterium tuberculosis and the implication for transmission in humans. Infect. Immun. 2002, 70, 4955-4960.
42. Torres, J. N.; Paul, L. V.; Rodwell, T. C.; Victor, T. C.; Amallraja, A. M.; Elghraoui, A.; Goodmanson, A. P.; Ramirez-Busby, S. M.; Chawla, A.; Zadorozhny, V.; et al. Novel katG mutations causing isoniazid resistance in clinical M. tuberculosis isolates. Emerg. Microbes Infect. 2015, 4, e42.
43. Yu, S.; Girotto, S.; Lee, C.; Magliozzo, R. S. Reduced affinity for Isoniazid in the S315T mutant of Mycobacterium tuberculosis KatG is a key factor in antibiotic resistance. J. Biol. Chem. 2003, 278, 14769-14775.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

What is claimed is:

1. A SuperSelective primer specific for a mutant target sequence of one selected from the group consisting of a katG gene, an inhA promoter, or an rpoB gene of Mycobacterium tuberculosis, comprising one or more target-complementary sequences that are at least 85% identical to one selected from the group consisting of SEQ ID NOs: 7-78.

2. The SuperSelective primer of claim 1, comprising, in the 5′ to 3′ direction, the following three contiguous DNA sequences: an anchor sequence capable of forming a hybrid with the mutant target sequence; a bridge sequence that is not complementary to either the mutant target sequence or the wild-type target sequence thereof; and a foot sequence that is perfectly complementary to the mutant target sequence and mismatched to the wild-type target sequence, wherein, if the anchor sequence and the foot sequence are hybridized either to the mutant target sequence or to the wild-type target sequence, there is in the target sequence an intervening sequence that does not hybridize to the primer's bridge sequence, and the bridge sequence and the intervening sequence together create a single-stranded bubble in the hybrid.

3. The SuperSelective primer of claim 2, wherein the foot sequence is mismatched to the wild-type target sequence by at least one of the 3′ nucleotide or the 3′ penultimate nucleotide of the foot sequence.

4. The SuperSelective primer of claim 1, wherein the mutant target sequence comprises a mutation selected from the group consisting of katG AGC→ACA, katG AGC→ACC, inhA −8T→A, inhA −15C→T, inhA −17G→T, rpoB GAC→GTC, rpoB CAC→GAC, rpoB CAC→TAC and rpoB TCG→TTG.

5. A method for detecting a drug-resistance mutation in M. tuberculosis or drug-resistant M. tuberculosis, comprising: providing a first primer pair specific for a first target segment of a first region selected from the group consisting of a katG gene, an inhA promoter, and an rpoB gene, said first primer pair comprising a first forward primer and a first reverse primer, wherein the first forward primer or the first reverse primer comprises the SuperSelective primer of claim 1; forming a reaction mixture comprising (i) the first primer pair and (ii) a sample comprising a nucleic acid of M. tuberculosis; amplifying the first target segment in the reaction mixture with the first primer pair to generate a first amplicon, and detecting the first amplicon, whereby the presence of the first amplicon is indicative of the drug resistance or drug-resistant M. tuberculosis in the sample.

6. The method of claim 5, wherein amplification is carried out by a polymerase chain reaction (PCR).

7. The method of claim 6, wherein the PCR is a real-time PCR, a quantitative PCR, or a non-symmetric PCR.

8. The method of claim 5, wherein amplification is carried out in the presence of a detection agent.

9. The method of claim 8, wherein the detection agent is a double-stranded DNA binding dye.

10. The method of claim 8, wherein the detection agent is a probe.

11. The method of claim 8, wherein the probe is labelled with a fluorophore.

12. The method of claim 11, wherein the probe is labelled with a fluorophore and a quencher.

13. The method of claim 12, wherein the fluorophore is selected from the group consisting of FAM, Cal Fluor Red, Quasar 670, fluorescein, cyanine 3, cyanine 5, Texas Red, and TAMRA.

14. The method of claim 12, wherein the quencher is selected from the group consisting of BHQ1, BHQ2, and DABCYL.

15. The method of claim 5, further comprising amplifying a second target segment with a second primer pair to generate a second amplicon, the second primer pair being specific for the second target segment.

16. The method of claim 5, wherein the resistance is to a drug selected from the group consisting of isoniazid, rifampicin, the fluoroquinolone class of drugs, amikacin, kanamycin, capreomycin, and ethambutol.

17. A probe comprising the nucleotide sequence of one selected from the group consisting of SEQ ID NOs: 1-6.

18. The probe of claim 17, wherein the probe is labeled.

19. A reaction mixture composition or a kit comprising the SuperSelective primer of claim 1.

20. The reaction mixture composition or kit of claim 19 further comprising one or more of: (i) an amplification primer for the katG gene, the inhA promoter, or the rpoB gene, (ii) a probe specific for the katG gene, the inhA promoter, or the rpoB gene, (iii) a DNA polymerase, (iv) nucleoside triphospates, (v) a double-stranded DNA binding dye, (vi) a selectivity-enhancing reagent, and (vii) a buffer.
